(19) United States
(12) Patent Application Publication
(10) Pub. No.: US 2010/0184747 A1
(43) Pub. Date: Jul. 22, 2010

(54) INDOLINE DERIVATIVES AND THEIR USE IN TREATING DISEASE-STATES SUCH AS CANCER

(75) Inventors: Matthias Treu, Vienna (AT); Ulrich Guertler, Vienna (AT); Thomas Karner, Vienna (AT); Oliver Kraemer, Vienna (AT); Jens Juergen Quant, Perchtoldsdorf (AT); Stephan Karl Zahn, Vienna (AT)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/663,876

(22) PCT Filed: Jun. 9, 2008
(86) PCT No.: PCT/EP08/57149

§ 371 Date: Mar. 15, 2010

(30) Foreign Application Priority Data
Jun. 12, 2007 (EP) 07110047.3

(51) Int. Cl. A61K 31/551; A61P 35/04; A61P 31/00; A61P 29/00; C07D 403/12; A61K 31/4184; C07D 401/14; A61K 31/4439; C07D 403/14; A61K 31/497; C07D 413/14; A61K 31/5377

(52) U.S. Cl. 514/218; 548/305.1; 514/394; 546/273.4; 514/338; 540/575; 544/370; 514/254.06; 544/139; 514/234.5

(57) ABSTRACT

The present invention encompasses compounds of general formula (1) wherein R¹ to R⁴ are defined as in claim 1, which are suitable for the treatment of diseases characterised by excessive or abnormal cell proliferation, and their use for preparing a pharmaceutical composition having the above-mentioned properties.

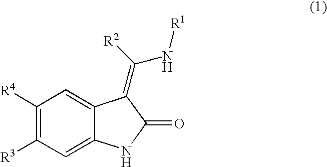

The present invention relates to new indolinones of general formula (1)

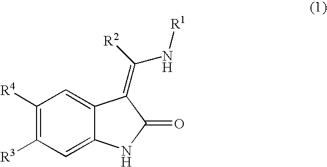

wherein the groups R¹ to R⁴ have the meanings given in the claims and specification, the isomers thereof, processes for preparing these indolinones and their use as medicaments.

The aim of the present invention is to discover new active substances which can be used for the prevention and/or treatment of diseases characterised by excessive or abnormal cell proliferation.

### BACKGROUND TO THE INVENTION

Indolinones are described for example as receptor tyrosinekinases and cyclin/CDK-complex inhibiting compounds, and are substituted in the 6 position either with a methyl carboxylate (WO02/081445), carbamoyl (WO01/27081) or with halogens (WO2004/026829).

### DETAILED DESCRIPTION OF THE INVENTION

It has now been found that, surprisingly, compounds of general formula (1), wherein the groups R¹ to R⁴ have the meanings given hereinafter act as inhibitors of specific cell cycle kinases. Thus, the compounds according to the invention may be used for example for the treatment of diseases connected with the activity of specific cell cycle kinases and characterised by excessive or abnormal cell proliferation.

The present invention relates to compounds of general formula (1)

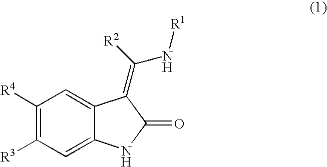

wherein
R¹ denotes hydrogen or a group, optionally substituted by one or more R⁵, selected from among C₃₋₁₀cycloalkyl, 3-8 membered heterocycloalkyl, C₆₋₁₅aryl and 5-15 membered heteroaryl; and
R² denotes a group, optionally substituted by one or more R⁵, selected from among C₆₋₁₅aryl and 5-15 membered heteroaryl; and
R³ denotes a group, optionally substituted by one or more R⁵, selected from among 3-8 membered heterocycloalkyl and 5-12 membered heteroaryl, or —N(R^{g})C(O)R^{c}, —N(R^{g})S(O)₂R^{c}, —N(R^{g})S(O)₂NR^{c}R^{c}, —N(R⁹)[C(O)]₂NR^{c}R^{c}, —N(R^{g})C(O)OR^{c}, and
R⁴ denotes hydrogen or a group selected from among halogen, —CN, —OR^{e}, —NR^{e}R^{e} and C₁₋₆alkyl, and
R⁵ in each case independently of one another denote a group selected from among R^{a}, R^{b} and R^{a} substituted by one or more identical or different R^{b} and/or R^{c}; and
each R^{a} independently of one another is selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl;

each R^{b} is a suitable group and each is independently selected from among ═O, —OR^{c}, C₁₋₃haloalkyloxy, —OCF₃, ═S, —SR^{C}, ═NR^{C}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g})[C(O)]₂OR^{c}, —N(R^{g})[C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c},

each R^{c} independently of one another denotes hydrogen or a group optionally substituted by one or more identical or different R^{d} and/or R^{e} selected from among C₁₋₆alkyl, C₃₋₁₀cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl;
each R^{d} is a suitable group and each is independently selected from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}—N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e}}₂, —N(R^{g})[C(O)]₂OR^{e}, —N(R^{g})[C(O)]₂NR^{e}R^{e}, —N{[C(O)]₂OR^{e}}₂, —N{[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e} and —N(R^{g})C(NR^{g})NR^{e}R^{e},
each R^{e} independently of one another denotes hydrogen or a group optionally substituted by one or more identical or different R^{f} and/or R^{g} selected from among C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl;
each R^{f} is a suitable group and each is independently selected from among halogen and —CF₃; and
each R^{g} independently of one another denotes hydrogen, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkyl, 5-12 membered heteroaryl or 6-18 membered heteroarylalkyl, optionally in the form of the prodrugs, the tautomers, the racemates, the enantiomers, the diastereomers and the mixtures thereof, and optionally the pharmacologically acceptable acid addition salts thereof with the proviso that 6-benzoylamino-3-(Z)-{1-[4-(piperidin-1yl-methyl)-anilino]-1-phenyl-methylidene}-2-indolinone, 3-(Z)-{1-[4-(piperidin-1-yl-methyl)-anilino]-1-phenyl-methylidene}-6-(pyrrol-1-yl)-2-indolinone and 3-(Z)-{1-[4-(piperidin-1-yl-methyl)-anilino]-1-phenyl-methylidene}-6-(pyrrolidin-1-yl)-2-indolinone are not included.

In one aspect the invention relates to compounds of general formula (1) wherein R⁴ is hydrogen.

In another aspect the invention relates to compounds of general formula (1) wherein R¹ denotes phenyl.

In another aspect the invention relates to compounds of general formula (1) wherein R² denotes phenyl.

In another aspect the invention relates to compounds of general formula (1) wherein R² denotes unsubstituted phenyl.

In another aspect the invention relates to compounds of general formula (1) wherein R³ denotes —N(R^{g})C(O)R^{c}.

In another aspect the invention relates to compounds of general formula (1) as pharmaceutical compositions.

In another aspect the invention relates to compounds of general formula (1) for preparing a pharmaceutical composition with an antiproliferative activity.

In another aspect the invention relates to a pharmaceutical preparation, containing as active substance one or more compounds of general formula (1) or the physiologically acceptable salts thereof, optionally in combination with conventional excipients and/or carriers.

In another aspect the invention relates to the use of compounds of general formula (1) for preparing a pharmaceutical composition for the treatment and/or prevention of cancer, infections, inflammations and autoimmune diseases.

In another aspect the invention relates to a pharmaceutical preparation comprising a compound of general formula (1) and at least one further cytostatic or cytotoxic active substance, different from formula (1), optionally in the form of the tautomers, the racemates, the enantiomers, the diastereomers and the mixtures thereof, and optionally the pharmacologically acceptable acid addition salts thereof.

### DEFINITIONS

As used herein, the following definitions apply, unless stated otherwise.

Alkyl is made up of the sub-groups saturated hydrocarbon chains and unsaturated hydrocarbon chains, while the latter may be further subdivided into hydrocarbon chains with a double bond (alkenyl) and hydrocarbon chains with a triple bond (alkynyl). Alkenyl contains at least one double bond, alkynyl at least one triple bond. If a hydrocarbon chain should have both at least one double bond and at least one triple bond, by definition it belongs to the alkynyl sub-group. All the above-mentioned sub-groups may be further subdivided into straight-chain (unbranched) and branched. If an alkyl is substituted, it may be mono- or polysubstituted independently of one another at all the hydrogen-carrying carbon atoms.

Examples of individual sub-groups are listed below.

Straight-chain (unbranched) or branched, saturated hydrocarbon chains:

methyl; ethyl; n-propyl; isopropyl (1-methylethyl); n-butyl; 1-methylpropyl; isobutyl (2-methylpropyl); sec.-butyl (1-methylpropyl); tent.-butyl (1.1-dimethylethyl); n-pentyl; 1-methylbutyl; 1-ethylpropyl; isopentyl (3-methylbutyl); neopentyl (2,2-dimethyl-propyl); n-hexyl; 2,3-dimethylbutyl; 2,2-dimethylbutyl; 3,3-dimethylbutyl; 2-methyl-pentyl; 3-methylpentyl; n-heptyl; 2-methylhexyl; 3-methylhexyl; 2,2-dimethylpentyl; 2,3-dimethylpentyl; 2,4-dimethylpentyl; 3,3-dimethylpentyl; 2,2,3-trimethylbutyl; 3-ethylpentyl; n-octyl; n-nonyl; n-decyl etc.

straight-chained (unbranched) or branched alkenyl:

vinyl (ethenyl); prop-1-enyl; allyl (prop-2-enyl); isopropenyl; but-1-enyl; but-2-enyl; but-3-enyl; 2-methyl-prop-2-enyl; 2-methyl-prop-1-enyl; 1-methyl-prop-2-enyl; 1-methyl-prop-1-enyl; 1-methylidenepropyl; pent-1-enyl; pent-2-enyl; pent-3-enyl; pent-4-enyl; 3-methyl-but-3-enyl; 3-methyl-but-2-enyl; 3-methyl-but-1-enyl; hex-1-enyl; hex-2-enyl; hex-3-enyl; hex-4-enyl; hex-5-enyl; 2,3-dimethyl-but-3-enyl; 2,3-dimethyl-but-2-enyl; 2-methylidene-3-methylbutyl; 2,3-dimethyl-but-1-enyl; hexa-1,3-dienyl; hexa-1,4-dienyl; penta-1,4-dienyl; penta-1,3-dienyl; buta-1,3-dienyl; 2,3-dimethylbuta-1,3-diene etc.

straight-chain (unbranched) or branched alkynyl:

ethynyl; prop-1-ynyl; prop-2-ynyl; but-1-ynyl; but-2-ynyl; but-3-ynyl; 1-methyl-prop-2-ynyl etc.

By the terms propyl, butyl, pentyl, hexyl, heptyl, octyl, nonyl, decyl etc. unless otherwise stated are meant saturated hydrocarbon groups with the corresponding number of carbon atoms, including all the isomeric forms.

By the terms propenyl, butenyl, pentenyl, hexenyl, heptenyl, octenyl, nonenyl, decenyl etc. unless otherwise stated are meant unsaturated hydrocarbon groups with the corresponding number of carbon atoms and a double bond, including all the isomeric forms, also (Z)/(E)-isomers, where applicable.

By the terms butadienyl, pentadienyl, hexadienyl, heptadienyl, octadienyl, nonadienyl, decadienyl etc. unless otherwise stated are meant unsaturated hydrocarbon groups with the corresponding number of carbon atoms and two double bonds, including all the isomeric forms, also (Z)/(E)-isomers, where applicable.

By the terms propynyl, butynyl, pentynyl, hexynyl, heptynyl, octynyl, nonynyl, decynyl etc. unless otherwise stated are meant unsaturated hydrocarbon groups with the corresponding number of carbon atoms and a triple bond, including all the isomeric forms.

By the term heteroalkyl are meant groups which are derived from the alkyl as hereinbefore defined in its widest sense by replacing, in the hydrocarbon chains, one or more of the groups —CH₃ independently of one another by the groups —OH, —SH or —NH₂, one or more of the groups —CH₂— independently of one another by the groups —O—, —S— or —NH—, one or more of the groups

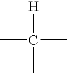

by the group

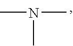

one or more of the groups ═CH— by the group ═N—, one or more of the groups ═CH₂ by the group ═NH or one or more of the groups ≡CH by the group ≡N, while a total of not more than three heteroatoms may be present in one heteroalkyl, there must be at least one carbon atom between two oxygen atoms and between two sulphur atoms or between one oxygen and one sulphur atom and the group as a whole must have chemical stability.

A direct result of the indirect definition/derivation from alkyl is that heteroalkyl is made up of the sub-groups saturated hydrocarbon chains with heteroatom(s), heteroalkenyl and heteroalkynyl, and it may be further subdivided into straight-chain (unbranched) and branched. If a heteroalkyl is substituted, it may be mono- or polysubstituted independently of one another at all the hydrogen-carrying oxygen, sulphur, nitrogen and/or carbon atoms. Heteroalkyl itself as a substituent may be attached to the molecule both through a carbon atom and through a heteroatom.

The following are listed by way of example:

dimethylaminomethyl; dimethylaminoethyl (1-dimethylaminoethyl; 2-dimethyl-aminoethyl); dimethylaminopropyl (1-dimethylaminopropyl, 2-dimethylaminopropyl, 3-dimethylaminopropyl); diethylaminomethyl; diethylaminoethyl (1-diethylaminoethyl, 2-diethylaminoethyl); diethylaminopropyl (1-diethylaminopropyl, 2-diethylamino-propyl, 3-diethylaminopropyl); diisopropylaminoethyl (1-diisopropylaminoethyl, 2-di-isopropylaminoethyl); bis-2-methoxyethylamino; [2-(dimethylamino-ethyl)-ethyl-amino]-methyl; 3-[2-(dimethylamino-ethyl)-ethyl-amino]-propyl; hydroxymethyl; 2-hydroxy-ethyl; 3-hydroxypropyl; methoxy; ethoxy; propoxy; methoxymethyl; 2-methoxyethyl etc.

Halogen encompasses fluorine, chlorine, bromine and/or iodine atoms.

Haloalkyl is derived from alkyl as hereinbefore defined in its broadest sense, by replacing one or more hydrogen atoms of the hydrocarbon chain independently of one another by halogen atoms, which may be identical or different. A direct result of the indirect definition/derivation from alkyl is that haloalkyl is made up of the sub-groups saturated hydrohalogen chains, haloalkenyl and haloalkynyl, and it may be further subdivided into straight-chain (unbranched) and branched. If a haloalkyl is substituted, it may be mono- or polysubstituted independently of one another at all the hydrogen-carrying carbon atoms. Typical examples include, for example:

—CF₃; —CHF₂; —CH₂F; —CF₂CF₃; —CHFCF₃; —CH₂CF₃; —CF₂CH₃; —CHFCH₃; —CF₂CF₂CF₃; —CF₂CH₂CH₃; —CF═CF₂; CCl═CH₂; CBr═CH₂; —CI═CH₂; —C≡C—CF₃; —CHFCH₂CH₃; and —CHFCH₂CF₃.

Cycloalkyl is made up of the sub-groups monocyclic hydrocarbon rings, bicyclic hydrocarbon rings and spirohydrocarbon rings, while each sub-group may be further subdivided into saturated and unsaturated (cycloalkenyl). By unsaturated is meant that there is at least one double bond in the ring system, but no aromatic system is formed. In bicyclic hydrocarbon rings two rings are linked such that they share at least two carbon atoms. In spirohydrocarbon rings one carbon atom (spiroatom) is shared by two rings. If a cycloalkyl is substituted, it may be mono- or polysubstituted independently of one another at all the hydrogen-carrying carbon atoms. Cycloalkyl itself as a substituent may be attached to the molecule through any suitable position of the ring system. The following individual sub-groups are listed by way of example:

monocyclic saturated hydrocarbon rings:

cyclopropyl; cyclobutyl; cyclopentyl; cyclohexyl; cycloheptyl etc.

monocyclic unsaturated hydrocarbon rings:

cycloprop-1-enyl; cycloprop-2-enyl; cyclobut-1-enyl; cyclobut-2-enyl; cyclopent-1-enyl; cyclopent-2-enyl; cyclopent-3-enyl; cyclohex-1-enyl; cyclohex-2-enyl; cyclohex-3-enyl; cyclohept-1-enyl; cyclohept-2-enyl; cyclohept-3-enyl; cyclohept-4-enyl; cyclobuta-1,3-dienyl; cyclopenta-1,4-dienyl; cyclopenta-1,3-dienyl; cyclopenta-2,4-dienyl; cyclohexa-1,3-dienyl; cyclohexa-1,5-dienyl; cyclohexa-2,4-dienyl; cyclohexa-1,4-dienyl; cyclohexa-2,5-dienyl etc.

saturated and unsaturated bicyclic hydrocarbon rings:

bicyclo[2.2.0]hexyl; bicyclo[3.2.0]heptyl; bicyclo[3.2.1]octyl; bicyclo[2.2.2]octyl; bicyclo[4.3.0]nonyl (octahydroindenyl); bicyclo[4.4.0]decyl (decahydronaphthalene); bicyclo[2.2.1]heptyl (norbornyl); (bicyclo[2.2.1]hepta-2,5-dienyl (norborna-2,5-dienyl); bicyclo[2.2.1]hept-2-enyl (norbornenyl); bicyclo[4.1.0]heptyl (norcaranyl); bicyclo-[3.1.1]heptyl (pinanyl) etc.

saturated and unsaturated spirohydrocarbon rings:

spiro[2.5]octyl, spiro[3.3]heptyl, spiro[4.5]dec-2-ene, etc.

Cycloalkylalkyl denotes the combination of the alkyl and cycloalkyl groups defined hereinbefore, in each case in their broadest sense. The alkyl group as substituent is directly linked to the molecule and is in turn substituted by a cycloalkyl group. The linking of alkyl and cycloalkyl in both groups may be effected by means of any suitable carbon atoms. The sub-groups of alkyl and cycloalkyl are also included in the combination of the two groups.

Aryl denotes mono-, bi- or tricyclic carbon rings with at least one aromatic ring. If an aryl is substituted, the substitution may be mono- or polysubstitution in each case, at all the hydrogen-carrying carbon atoms, independently of one another. Aryl itself may be linked to the molecule as substituent via any suitable position of the ring system. Typical examples include phenyl, naphthyl, indanyl (2,3-dihydroindenyl), 1,2,3,4-tetrahydronaphthyl and fluorenyl.

Arylalkyl denotes the combination of the groups alkyl and aryl as hereinbefore defined, in each case in their broadest sense. The alkyl group as substituent is directly linked to the molecule and is in turn substituted by an aryl group. The alkyl and aryl may be linked in both groups via any carbon atoms suitable for this purpose. The respective sub-groups of alkyl and aryl are also included in the combination of the two groups.

Typical examples include benzyl; 1-phenylethyl; 2-phenylethyl; phenylvinyl; phenylallyl etc.

Heteroaryl denotes monocyclic aromatic rings or polycyclic rings with at least one aromatic ring, which, compared with corresponding aryl or cycloalkyl, contain instead of one or more carbon atoms one or more identical or different heteroatoms, selected independently of one another from among nitrogen, sulphur and oxygen, while the resulting group must be chemically stable. If a heteroaryl is substituted, the substitution may be mono- or polysubstitution in each case, at all the hydrogen-carrying carbon and/or nitrogen atoms, independently of one another. Heteroaryl itself as substituent may be linked to the molecule via any suitable position of the ring system, both carbon and nitrogen.

Typical examples are listed below.

monocyclic heteroaryls:

furyl; thienyl; pyrrolyl; oxazolyl; thiazolyl; isoxazolyl; isothiazolyl; pyrazolyl; imidazolyl; triazolyl; tetrazolyl; oxadiazolyl; thiadiazolyl; pyridyl; pyrimidyl; pyridazinyl; pyrazinyl; triazinyl; pyridyl-N-oxide; pyrrolyl-N-oxide; pyrimidinyl-N-oxide; pyridazinyl-N-oxide; pyrazinyl-N-oxide; imidazolyl-N-oxide; isoxazolyl-N-oxide; oxazolyl-N-oxide; thiazolyl-N-oxide; oxadiazolyl-N-oxide; thiadiazolyl-N-oxide; triazolyl-N-oxide; tetrazolyl-N-oxide etc.

polycyclic heteroaryls:

indolyl; isoindolyl; benzofuryl; benzothienyl; benzoxazolyl; benzothiazolyl; benzisoxazolyl; benzisothiazolyl; benzimidazolyl; indazolyl; isoquinolinyl; quinolinyl; quinoxalinyl; cinnolinyl; phthalazinyl; quinazolinyl; benzotriazinyl; indolizinyl; oxazolopyridyl; imidazopyridyl; naphthyridinyl; indolinyl; isochromanyl; chromanyl; tetrahydroisoquinolinyl; isoindolinyl; isobenzotetrahydrofuryl; isobenzotetrahydrothienyl; isobenzothienyl; benzoxazolyl; pyridopyridyl; benzotetrahydrofuryl; benzotetrahydro-thienyl; purinyl; benzodioxolyl; phenoxazinyl; phenothiazinyl; pteridinyl; benzothiazolyl; imidazopyridyl; imidazothiazolyl; dihydrobenzisoxazinyl; benzisoxazinyl; benzoxazinyl; dihydrobenzisothiazinyl; benzopyranyl; benzothiopyranyl; cumarinyl; isocumarinyl; chromonyl; chromanonyl; tetrahydroquinolinyl; dihydroquinolinyl; dihydroquinolinonyl; dihydroisoquinolinonyl; dihydrocumarinyl; dihydroisocumarinyl; isoindolinonyl; benzodioxanyl; benzoxazolinonyl; quinolinyl-N-oxide; indolyl-N-oxide; indolinyl-N-oxide; isoquinolyl-N-oxide; quinazolinyl-N-oxide; quinoxalinyl-N-oxide; phthalazinyl-N-oxide; indolizinyl-N-oxide; indazolyl-N-oxide; benzothiazolyl-N-oxide; benzimidazolyl-N-oxide; benzo-thiopyranyl-5-oxide and benzothiopyranyl-S,S-dioxide etc.

Heteroarylalkyl denotes the combination of the alkyl and heteroaryl groups defined hereinbefore, in each case in their broadest sense. The alkyl group as substituent is directly linked to the molecule and is in turn substituted by a heteroaryl group. The linking of the alkyl and heteroaryl may be achieved on the alkyl side via any carbon atoms suitable for this purpose and on the heteroaryl side by any carbon or nitrogen atoms suitable for this purpose. The respective sub-groups of alkyl and heteroaryl are also included in the combination of the two groups.

By the term heterocycloalkyl are meant groups which are derived from the cycloalkyl as hereinbefore defined if in the hydrocarbon rings one or more of the groups —CH₂— are replaced independently of one another by the groups —O—, —S— or —NH— or one or more of the groups ═CH— are replaced by the group ═N—, while not more than five heteroatoms may be present in total, there must be at least one carbon atom between two oxygen atoms and between two sulphur atoms or between one oxygen and one sulphur atom and the group as a whole must be chemically stable. Heteroatoms may simultaneously be present in all the possible oxidation stages (sulphur→sulphoxide —SO—, sulphone —SO₂—; nitrogen →N-oxide). It is immediately apparent from the indirect definition/derivation from cycloalkyl that heterocycloalkyl is made up of the sub-groups monocyclic hetero-rings, bicyclic hetero-rings and spirohetero-rings, while each sub-group can also be further subdivided into saturated and unsaturated (heterocycloalkenyl). The term unsaturated means that in the ring system in question there is at least one double bond, but no aromatic system is formed. In bicyclic hetero-rings two rings are linked such that they have at least two atoms in common. In spirohetero-rings one carbon atom (spiroatom) is shared by two rings. If a heterocycloalkyl is substituted, the substitution may be mono- or poly-substitution in each case, at all the hydrogen-carrying carbon and/or nitrogen atoms, independently of one another. Heterocycloalkyl itself as substituent may be linked to the molecule via any suitable position of the ring system.

Typical examples of individual sub-groups are listed below.

monocyclic heterorings (saturated and unsaturated):

tetrahydrofuryl; pyrrolidinyl; pyrrolinyl; imidazolidinyl; thiazolidinyl; imidazolinyl; pyrazolidinyl; pyrazolinyl; piperidinyl; piperazinyl; oxiranyl; aziridinyl; azetidinyl; 1,4-dioxanyl; azepanyl; diazepanyl; morpholinyl; thiomorpholinyl; homomorpholinyl; homopiperidinyl; homopiperazinyl; homothiomorpholinyl; thiomorpholinyl-5-oxide; thiomorpholinyl-S,S-dioxide; 1,3-dioxolanyl; tetrahydropyranyl; tetrahydrothiopyranyl; [1,4]-oxazepanyl; tetrahydrothienyl; homothiomorpholinyl-S,S-dioxide; oxazolidinonyl; dihydropyrazolyl; dihydropyrrolyl; dihydropyrazinyl; dihydropyridyl; dihydro-pyrimidinyl; dihydrofuryl; dihydropyranyl; tetrahydrothienyl-5-oxide; tetrahydrothienyl-S,S-dioxide; homothiomorpholinyl-5-oxide; 2,3-dihydroazet; 2H-pyrrolyl; 4H-pyranyl; 1,4-dihydropyridinyl etc.

bicyclic heterorings (saturated and unsaturated):

8-azabicyclo[3.2.1]octyl; 8-azabicyclo[5.1.0]octyl; 2-oxa-5-azabicyclo[2.2.1]heptyl; 8-oxa-3-aza-bicyclo[3.2.1]octyl; 3,8-diaza-bicyclo[3.2.1]octyl; 2,5-diaza-bicyclo-[2.2.1]heptyl; 1-aza-bicyclo[2.2.2]octyl; 3,8-diaza-bicyclo[3.2.1]octyl; 3,9-diaza-bicyclo[4.2.1]nonyl; 2,6-diaza-bicyclo[3.2.2]nonyl; hexahydro-furo[3,2-b]furyl; etc.

spiro-heterorings (saturated and unsaturated):

1,4-dioxa-spiro[4.5]decyl; 1-oxa-3.8-diaza-spiro[4.5]decyl; and 2,6-diaza-spiro[3.3]heptyl; 2,7-diaza-spiro[4.4]nonyl; 2,6-diaza-spiro[3.4]octyl; 3,9-diaza-spiro[5.5]undecyl; 2,8-diaza-spiro[4.5]decyl etc.

Heterocycloalkylalkyl denotes the combination of the alkyl and heterocycloalkyl groups defined hereinbefore, in each case in their broadest sense. The alkyl group as substituent is directly linked to the molecule and is in turn substituted by a heterocycloalkyl group. The linking of the alkyl and heterocycloalkyl may be achieved on the alkyl side via any carbon atoms suitable for this purpose and on the heterocycloalkyl side by any carbon or nitrogen atoms suitable for this purpose. The respective sub-groups of alkyl and heterocycloalkyl are also included in the combination of the two groups.

By the term “suitable substituent” is meant a substituent which on the one hand is suitable by virtue of its valency and on the other hand leads to a system which is chemically stable.

By “prodrug” is meant an active substance in the form of its precursor metabolite. A distinction may be made between partly multi-part carrier-prodrug systems and bio-transformation systems. The latter contain the active active substance in a form that requires chemical or biological metabolisation. The skilled man will be familiar with prodrug systems of this kind (Sloan, Kenneth B.; Wasdo, Scott C. The role of prodrugs in penetration enhancement. Percutaneous Penetration Enhancers (2nd Edition) (2006), 51-64; Lloyd, Andrew W. Prodrugs. Smith and Williams' Introduction to the Principles of Drug Design and Action (4th Edition) (2006), 211-232; Neervannan, Seshadri. Strategies to impact solubility and dissolution rate during drug lead optimization: salt selection and prodrug design approaches. American Pharmaceutical Review (2004), 7(5), 108.110-113).

A suitable prodrug contains for example a substance of the general formulae which is linked via an enzymatically cleavable linker (e.g. carbamate, phosphate, N-glycoside or a disulphide group to a dissolution-improving substance (e.g. tetraethyleneglycol, saccharide, amino acids). Carrier-prodrug systems contain the active substance as such, bound to a masking group which can be cleaved by the simplest possible controllable mechanism. The function of masking groups according to the invention in the compounds according to the invention is to neutralise the charge for improving cell uptake. If the compounds according to the invention are used with a masking group, these may also additionally influence other pharmacological parameters, such as for example oral bioavailability, tissue distribution, pharmacokinetics and stability against non-specific phosphatases. The delayed release of the active substance may also involve a sustained-release effect. In addition, modified metabolisation may occur, thus resulting in a higher efficiency of the active substance or organic specificity. In the case of a prodrug formulation, the masking group or a linker that binds the masking group to the active substance is selected such that the prodrug is sufficienyl hydrophilic to be dissolved in the blood serum, has sufficient chemical and enzymatic stability to reach the activity site and is also sufficiently hydrophilic to ensure that it is suitable for diffusion-controlled membrane transport. Furthermore, it should allow chemically or ensymatically induced release of the active substance within a reasonable period and, it goes without saying, the auxiliary components released should be non-toxic. Within the scope of the invention, however, the compound without a mask or linker, and a mask, may be regarded as a prodrug which first of all has to be prepared in the cell from the ingested compound by enzymatic and biochemical processes.

### Preparation of the Compounds According to the Invention 6-Nitroindolinones

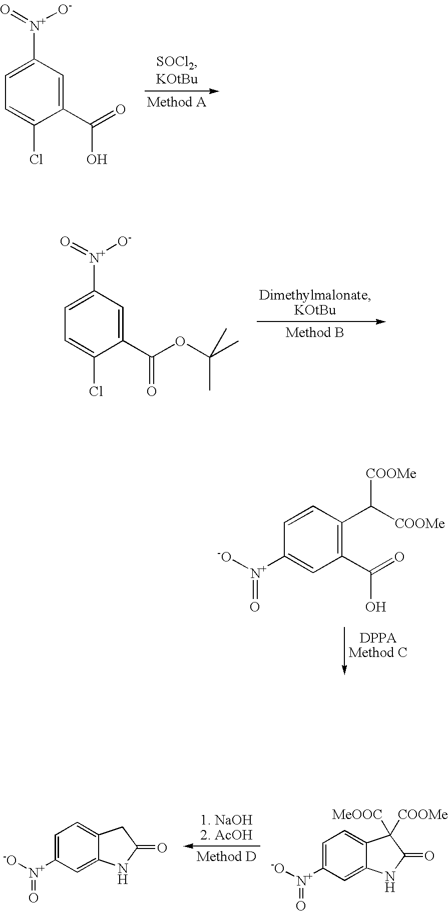

Method A—tert. Butyl 2-chloro-4-nitrobenzenecarboxylate (Z1)

2-Chloro-5-nitrobenzoic acid (22 g, 109.1 mmol) and DMF (500 μL) are refluxed in toluene (50 mL)/thionyl chloride (8.5 mL) for 1.5 h with stirring. The reaction mixture is evaporated down and the residue is taken up in anhydrous THF (200 mL). Potassium-tert.-butoxide (12.5 g, 111.4 mmol) is added at 0° C., then the cooling is removed and the mixture is stirred for 30 min The solvent is distilled off and the residue is divided between water and EtOAc. The organic phase is washed with water and 0.1 N NaOH, dried, filtered and evaporated down. Yield: 24 g (85%)

Method B—Dimethyl 2-(2-carboxy-4-nitrophenyl)malonate (Z2)

Potassium-tert.-butoxide (50 g, 446 mmol) is dissolved at 20° C. in anhydrous DMSO (300 mL), at this temperature dimethyl malonate (67 mL, 586 mmol) is added and the mixture is stirred for 20 min Z1 (45.7 g, 177 mmol) is added and the mixture is stirred for 30 min at 100° C. It is poured onto water (800 mL), acidified with concentrated HCl (30 mL) and extracted exhaustively with CH₂Cl₂. The organic phase is washed with water, dried, filtered and evaporated down. The residue is stirred in formic acid (300 mL) for 1.5 h at 72° C. The mixture is evaporated down, the residue is taken up in EtOAc, washed with NaCl solution and exhaustively extracted with dilute NaHCO₃ solution. The combined aqueous phase is acidified with concentrated HCl and exhaustively extracted with CH₂Cl₂. The combined organic phase is washed with water, dried, filtered and evaporated down. Yield: 38.4 g (73%)

Method C—Dimethyl 6-nitro-2-oxo-1,2-dihydroindol-3,3-dicarboxylate (Z3)

Triethylamine (9.4 mL, 67.8 mmol) is added to Z2 (20 g, 67.3 mmol) and DPPA (14.5 mL, 67.4 mmol) in anhydrous THF (40 mL) and the mixture is stirred for 1.25 h at boiling temperature. The reaction mixture is evaporated down, the residue is taken up in CH₂Cl₂ and washed with 1 N HCl. The organic phase is combined with ether and the precipitate is filtered off. Yield: 9.89 g (50%)

Method D—6-Nitro-1,3-dihydroindol-2-one (Z4)

Z3 (5.30 g, 10 mmol) is stirred in MeOH (10 mL)/2 N NaOH (10 mL) for 30 min at 80° C. The reaction mixture is acidified with 1 N HCl, the precipitate is filtered off and stirred in acetic acid (10 mL) for 1 h at boiling temperature. The mixture is cooled to RT, the precipitate is isolated by filtration and digested with water. Yield: 2.18 g (68%)

### Phenylenediamine Components

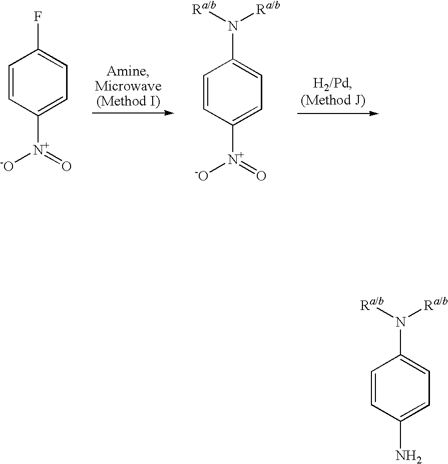

### Method I—Nucleophilic Aromatic Substitution

4-Fluoronitrobenzene (3 g, 21.3 mmol), 1-(1-methylpiperidin-4-yl)piperazine (3.90 g, 21.2 mmol) and triethylamine (3.30 mL, 23.7 mmol) are stirred in anhydrous isopropanol (10 mL) for 10 min at 160° C. in the microwave. The reaction mixture is diluted with water (10 mL), the precipitate is filtered off, washed with 50% water in isopropanol and dried in vacuo at 45° C. Yield: 5.14 g (79%)

If no crystalline product is obtained, the crude mixture is evaporated down, worked up by extraction and optionally purified by chromatography.

| | | | | Yield |
|---|---|---|---|---|
| # | Structure | Educt | Method | [%] |
| Z5 | 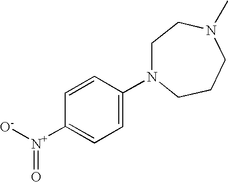 | 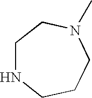 | I | 46 |
| Z6 | 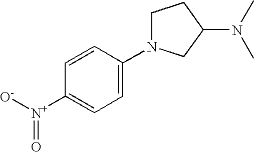 | 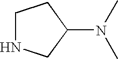 | I | 91 |
| Z7 | 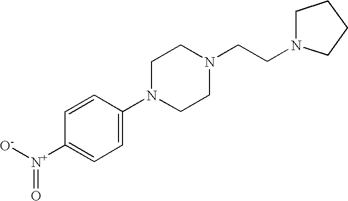 | 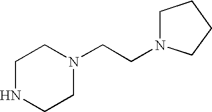 | I | 47 |
| Z8 | 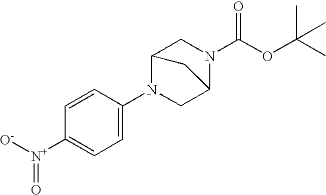 | 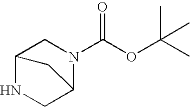 | I | 53 |
| Z9 | 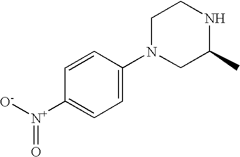 | 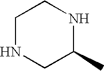 | I | 62 |
| Z10 | 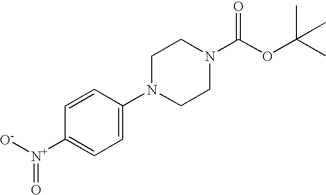 | 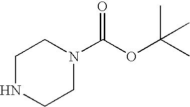 | I | 82 |
| Z11 | 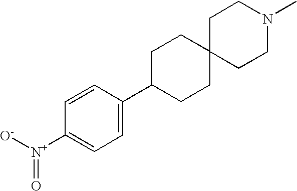 | 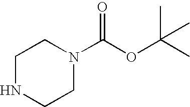 | I | 83 |
| Z12 | 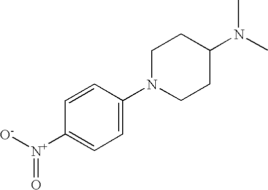 | 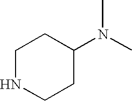 | I | 82 |
| Z13 | 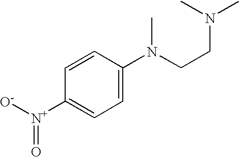 | 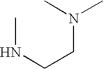 | I | 58 |
| Z14 | 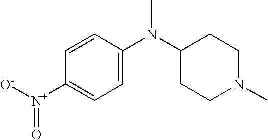 | 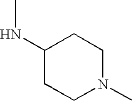 | I | 64 |

### Method R—Cleaving the Boc-Protective Group

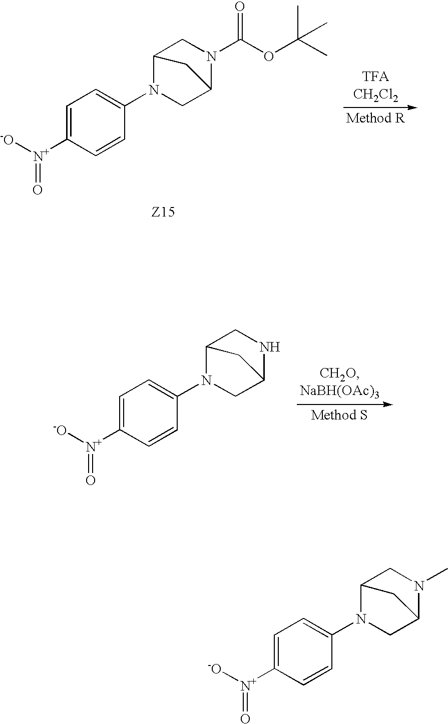

Z18 (2.80 g, 8.77 mmol) is stirred in CH₂Cl₂ (5 mL)/TFA (5 mL) for 30 min at 50° C. The reaction solution is diluted with CH₂Cl₂ and neutralised with K₂CO₃. The mixture is diluted with water and extracted exhaustively with EtOAc. The combined organic phases are dried, filtered and evaporated down. Yield: 1.60 g (83%)

### Method S—Reductive Amination

Z15 (1.60 g, 7.30 mmol) in CH₂Cl₂ (5 mL) and 37% formaldehyde in water (5 mL) are stirred for 1 h at RT. NaBH(OAc)₃ (4.95 g, 23.3 mmol) is added batchwise at 0° C., then the mixture is stirred for 3 h at RT. The reaction solution is divided between CH₂Cl₂ and saturated K₂CO₃ solution, the organic phase is washed with saturated K₂CO₃ solution, dried, filtered and evaporated down. Yield: 1.60 g (94%)

| | | | | Yield |
|---|---|---|---|---|
| # | Structure | Educt | Method | [%] |
| Z16 | 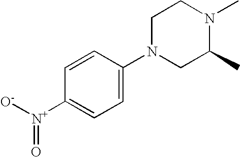 | 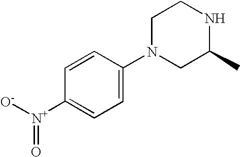 | S | 90 |

### Method J—Reduction of the Nitro Group

1-(1-methylpiperidin-4-yl)-4-(4-nitrophenyl)piperazine (5.14 g, 16.8 mmol) is dissolved in anhydrous THF (10 mL), combined with 10% palladium on activated charcoal and hydrogenated for 17 h at 3 bar hydrogen pressure at RT. More catalyst is metered in, if desired, and the hydrogen pressure is re-adjusted if it falls. The reaction mixture is filtered, evaporated down, combined with toluene (3×200 mL) and evaporated down again. Yield: 4.52 g (quant.)

| | | | | Yield |
|---|---|---|---|---|
| # | Structure | Educt | Method | [%] |
| Z17 | 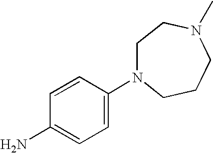 | 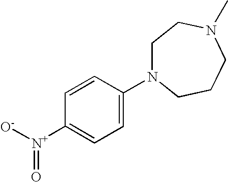 | J | quant. |
| Z18 | 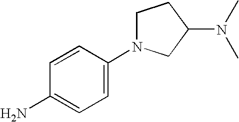 | 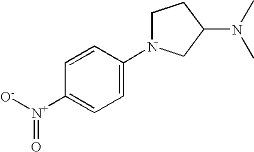 | J | quant. |
| Z19 | 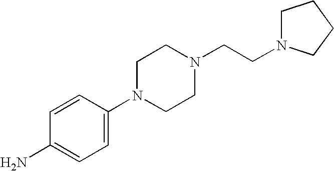 | 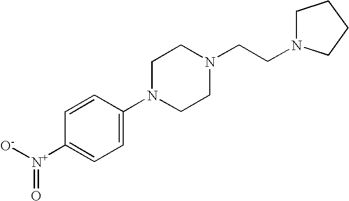 | J | 97 |
| Z20 | 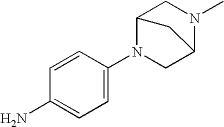 | 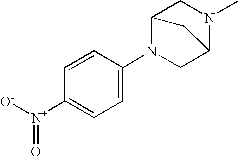 | J | 93 |
| Z21 | 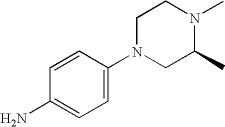 | 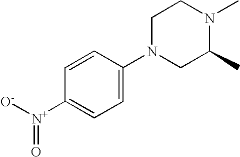 | J | 90 |
| Z22 | 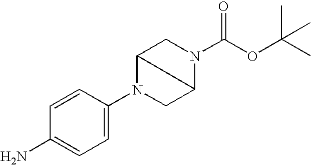 | 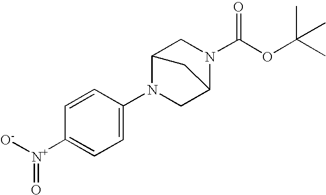 | J | 96 |
| Z23 | 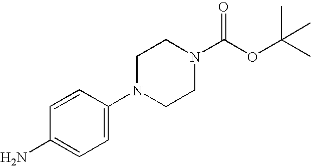 | 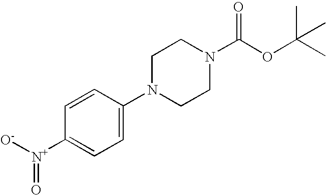 | J | 98 |
| Z24 | 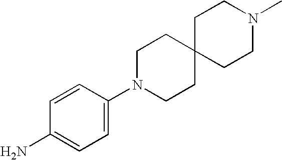 | 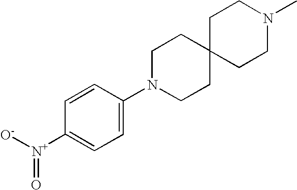 | J | 92 |
| Z25 | 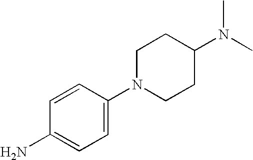 | 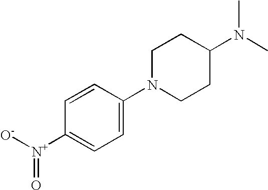 | J | 99 |
| Z26 | 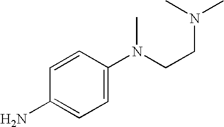 | 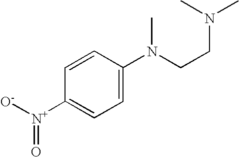 | J | 92 |
| Z27 | 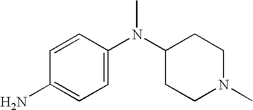 | 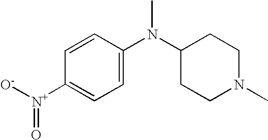 | J | 99 |

### Method T—Nucleophilic Aromatic Substitution

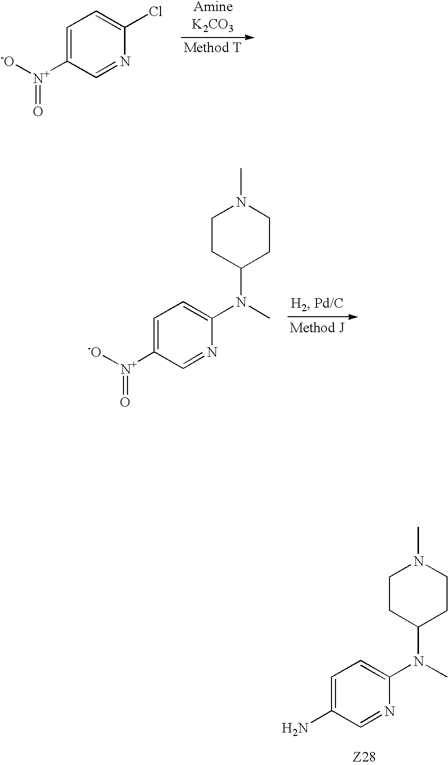

2-chloro-4-nitropyridin (2 g, 12.6 mmol), 1-methyl-4-methylaminopiperidine (1.83 mL, 12.6 mmol) and K₂CO₃ (2.62 g, 18.9 mmol) are stirred in dioxane (10 mL) for 16 h at 50° C. The reaction mixture is diluted with water and combined with saturated NH₄Cl solution. The aqueous phase is exhaustively extracted with CH₂Cl₂, the combined organic phases are dried, filtered and evaporated down. Yield: 2.65 g (84%)

| | | | | Yield |
|---|---|---|---|---|
| # | Structure | Educt | Method | [%] |
| Z29 | 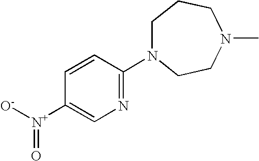 | 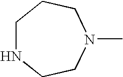 | T | 94 |
| Z30 | 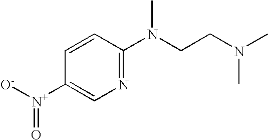 | 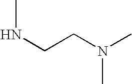 | T | 99 |
| Z31 | 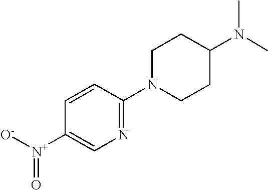 | 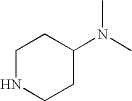 | T | quant. |
| Z32 | 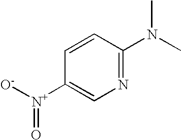 | 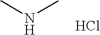 | T | 92 |

The reduction of the nitro group is carried out in 50% MeOH in THF according to Method J.

| | | | | Yield |
|---|---|---|---|---|
| # | Structure | Educt | Method | [%] |
| Z33 | 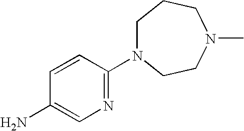 | 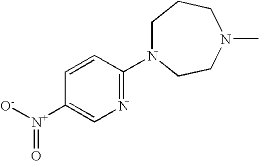 | J | quant. |
| Z34 | 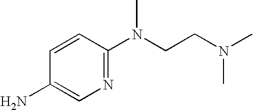 | 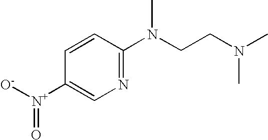 | J | 85 |
| Z35 | 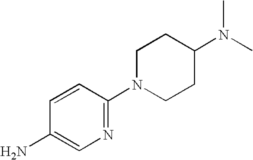 | 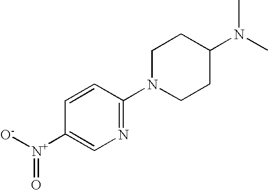 | J | quant. |
| Z36 | 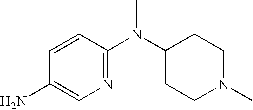 | 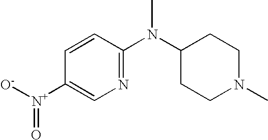 | J | 90 |
| Z37 | 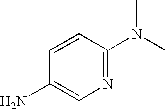 | 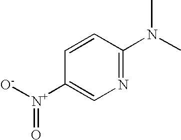 | J | quant |

### Preparation of the Benzylamine Components

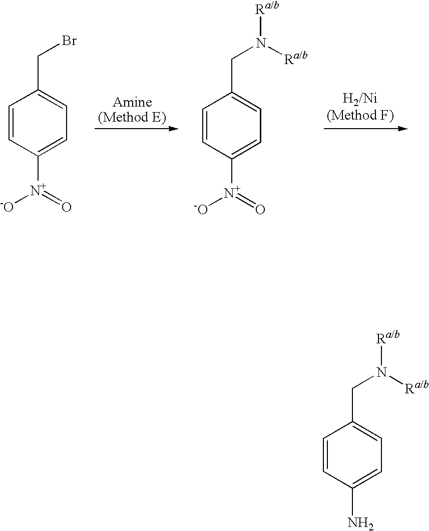

### Method E—1-(4-Nitrobenzyl)pyrrolidine (Z38)

A solution of pyrrolidine (24 mL, 290 mmol) in anhydrous THF (50 mL) is combined batchwise with 4-nitrobenzylbromide (25.00 g, 115 mmol) and stirred for 16 h at RT. The reaction mixture is evaporated down, taken up in EtOAc (300 mL), washed with saturated NH₄Cl solution, water and saturated saline solution, dried, filtered and evaporated down. Yield: 16.96 g (71%)

Alternatively potassium carbonate may be used as base.

| | | | | Yield |
|---|---|---|---|---|
| # | Structure | Educt | Method | [%] |
| Z39 | 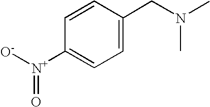 | 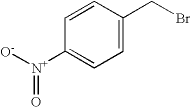 | E | 88 |
| Z40 | 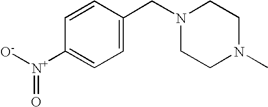 | 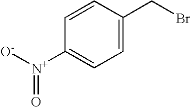 | E | 79 |
| Z41 | 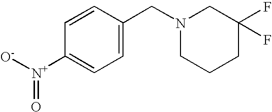 | 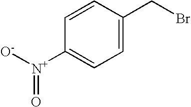 | E | 57 |
| Z42 | 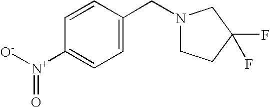 | 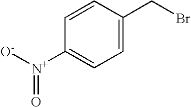 | E | 94 |

### Method F—Reduction of the Nitro Group

1-(4-Nitrobenzyl)pyrrolidine (16.96 g, 82.2 mmol) in anhydrous THF (50 mL) is combined with Raney nickel (5 g) and hydrogenated for 21 h under a hydrogen pressure of 7.5 bar at RT. More catalyst is metered in if desired and the hydrogen pressure is readjusted if it drops. The reaction mixture is filtered, evaporated down, combined with toluene (3×200 mL) and evaporated down again. Yield: 14.46 g (quant.)

| | | | | Yield |
|---|---|---|---|---|
| # | Structure | Educt | Method | [%] |
| Z43 | 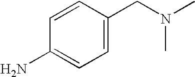 | 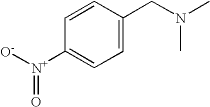 | F | 85 |
| Z44 | 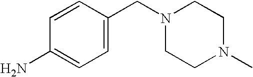 | 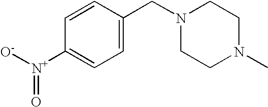 | F | 83 |
| Z45 | 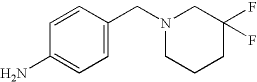 | 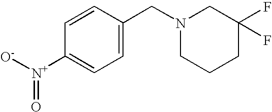 | F | 99 |
| Z46 | 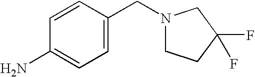 | 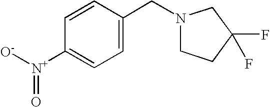 | F | quant. |

Method G—(2-Chloro-4-nitrophenyl)methanol (Z47)

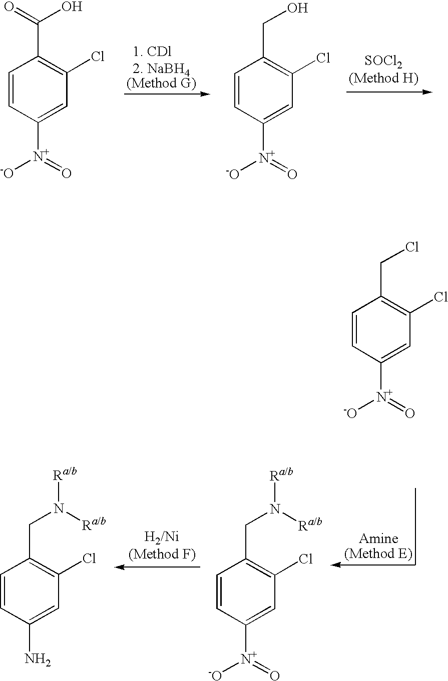

N,N′-Carbonyldiimidazole (19.91 g, 122 mmol) is added batchwise to 2-chloro-4-nitrobenzoic acid (25 g, 90% purity, 111 mmol) in anhydrous THF (420 mL) at RT and stirred for 1 h. At 15-20° C., NaBH (13.09 g, 346 mmol) in water (85 mL) is added dropwise thereto and the mixture is stirred for 16 h at RT. The reaction mixture is adjusted to pH 1 with 6 N HCl and exhaustively extracted with EtOAc. The combined organic phases are washed with 15% potassium carbonate solution (2×150 mL) and saturated saline solution (150 mL), dried, filtered and evaporated down. Yield: 20.60 g (98%)

| | | | | Yield |
|---|---|---|---|---|
| # | Structure | Educt | Method | [%] |
| Z48 | 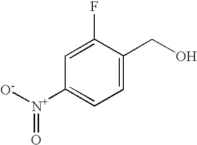 | 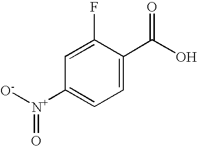 | G | 54 |
| Z49 | 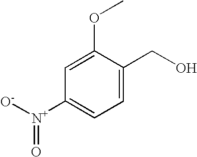 | 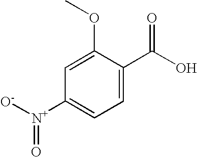 | G | 93 |

Method H—2-Chloro-1-chloromethyl-4-nitrobenzene (Z50)

(2-Chloro-4-nitrophenyl)methanol (19 g, 101 mmol) is stirred in a mixture of anhydrous DCM (400 mL), thionyl chloride (15 mL) and DMF (1 mL) for 2 h at boiling temperature. The reaction mixture is evaporated down, the residue is taken up in EtOAc (250 mL), washed with water (5×150 mL) and saturated saline solution (150 mL), dried, filtered and evaporated down. Yield: 20.40 g (98%)

| | | | | Yield |
|---|---|---|---|---|
| # | Structure | Educt | Method | [%] |
| Z51 | 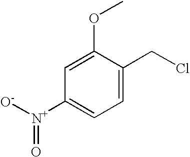 | 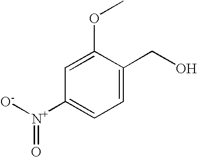 | H | 93 |

1-(2-Chloro-4-nitrobenzyl)pyrrolidine is prepared according to Method E.

| | | | | Yield |
|---|---|---|---|---|
| # | Structure | Educt | Method | [%] |
| Z52 | 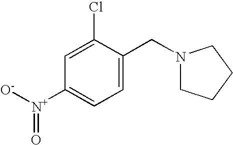 | 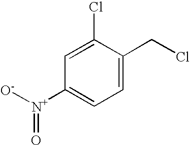 | E | 94 |
| Z53 | 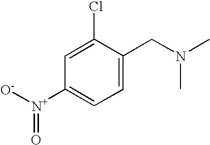 | 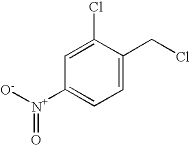 | E | 98 |
| Z54 | 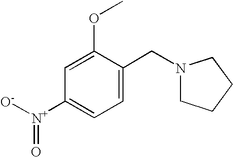 | 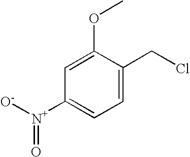 | E | 84 |

The reduction of the nitro group is carried out according to Method F.

| | | | | Yield |
|---|---|---|---|---|
| # | Structure | Educt | Method | [%] |
| Z55 | 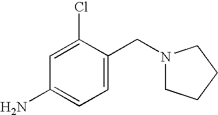 | 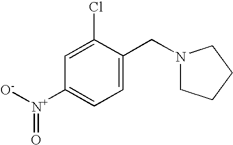 | F | 91 |
| Z56 | 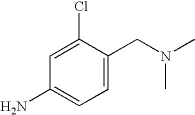 | 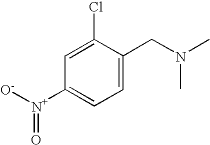 | F | quant. |
| Z57 | 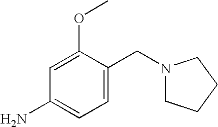 | 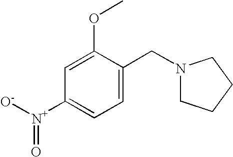 | F | 78 |

### Method U—Reductive Amination

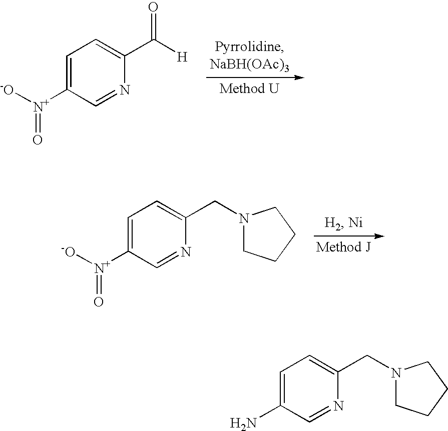

6-Nitropyridine-2-carbaldehyde (600 mg, 3.95 mmol) in anhydrous CH₂Cl₂ (2 mL) and pyrrolidine (391 μL, 4.73 mmol) are stirred for 15 min at RT. AcOH (371 μL) and NaBH(OAc)₃ (1.17 g, 5.52 mmol) are added and the mixture is stirred for 30 min at RT. The reaction solution is divided between CH₂Cl₂ and saturated NaHCO₃ solution, the organic phase is washed with saturated NaHCO₃ solution, dried, filtered and evaporated down. Yield: 850 mg (90%)

The reduction of the nitro group is carried out in MeOH according to Method J.

| | | | | Yield |
|---|---|---|---|---|
| # | Structure | Educt | Method | [%] |
| Z58 | 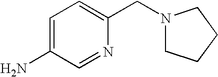 | 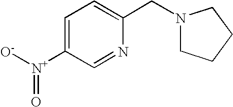 | J | quant. |

Method V—Reductive Amination with Formaldehyde

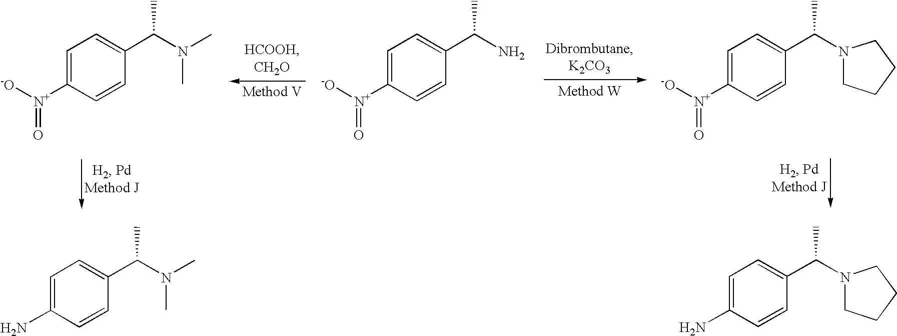

A solution of benzylamine (750 mg, 3.70 mmol) in 37% aqueous formaldehyde (1.3 mL) and HCOOH (1.55 mL) is stirred for 16 h at 100° C. The reaction solution is divided between CH₂Cl₂ and saturated K₂CO₃ solution, the organic phase is washed with saturated K₂CO₃ solution, dried, filtered and evaporated down. Yield: 682 mg (95%)

Method W—Alkylation with Dibromobutane

Benzylamine (2 g, 9.87 mmol), 1,4-dibromobutane (1.40 mL, 11.8 mmol), K₂CO₃ (4 g, 28.9 mmol) and KI (819 mg, 4.93 mmol) are refluxed in anhydrous MeCN for 16 h with stirring. The mixture is filtered, evaporated down and the residue is divided between water and CH₂Cl₂. The aqueous phase is exhaustively extracted with CH₂Cl₂. The combined organic phases are dried, filtered and evaporated down. Yield: 2.60 g (84%)

The reduction of the nitro group is carried out in THF according to Method J.

| | | | | Yield |
|---|---|---|---|---|
| # | Structure | Educt | Method | [%] |
| Z59 | 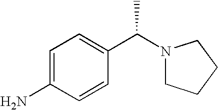 | 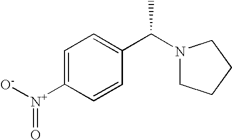 | J | 98 |
| Z60 | 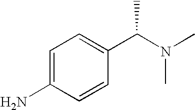 | 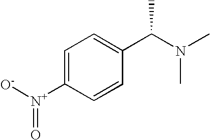 | J | 92 |

### Preparation of the Alkoxyaniline Components

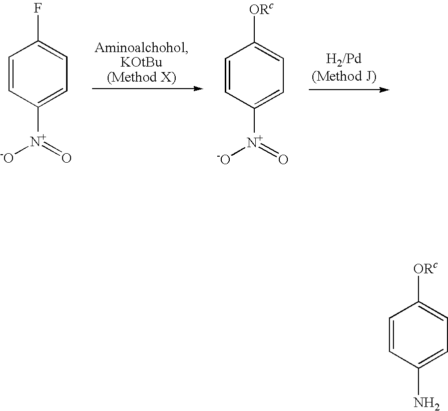

### Method X—Nucleophilic Aromatic Substitution (Z61)

4-Fluoronitrobenzene (2 mL, 18.9 mmol) is added to a solution of 4-hydroxy-1-methyl-piperidine (2.17 g, 18.9 mmol) and KOtBu (3.0 g, 26.7 mmol) in anhydrous DMSO (25 mL) and stirred for 2 h at RT. Water is added, the precipitate is isolated by filtration and the solid is dried in vacuo. Yield: 2.45 g (55%).

If no crystalline product is obtained the crude mixture is worked up by extraction and optionally purified by chromatography.

| | | | | Yield |
|---|---|---|---|---|
| # | Structure | Educt | Method | [%] |
| Z61 | 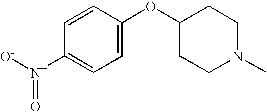 | 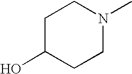 | X | 55 |
| Z62 | 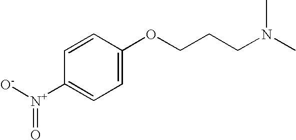 | 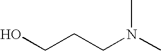 | X | 57 |
| Z63 | 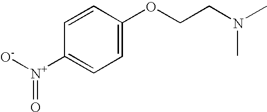 | 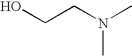 | X | 56 |

The reduction of the nitro group is carried out according to Method J.

| | | | | Yield |
|---|---|---|---|---|
| # | Structure | Educt | Method | [%] |
| Z64 | 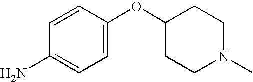 | 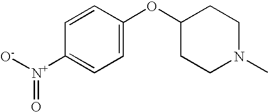 | J | 98 |
| Z65 | 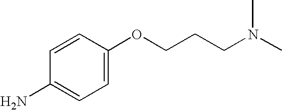 | 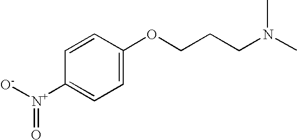 | J | 96 |
| Z66 | 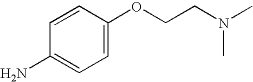 | 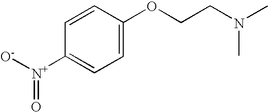 | J | 80 |

### Preparation of Phenylmethylidene-Indolinones

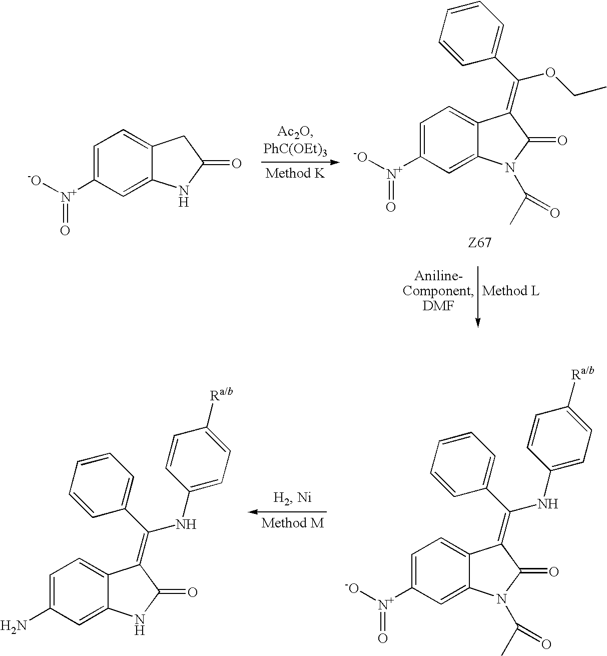

Method K—Condensation with Orthobenzoates

Z4 (2.18 g, 12.3 mmol) and triethyl orthobenzoate (8 mL, 35.2 mmol) is stirred in acetic anhydride (20 mL) for 10 min at 150° C. The mixture is cooled to RT, the precipitate is isolated by filtration and digested with water. Yield: 3.25 g (75%).

Method L—Substitution with Anilines

Z67 (2 g, 5.68 mmol) and 4-pyrrolidin-1-ylmethylphenylamine (1.05 g, 5.98 mmol) are stirred in anhydrous DMF (10 mL) for 2 h at 100° C. The mixture is cooled to RT, combined with H₂O/iPrOH=10/1 and the precipitate is isolated by filtration. Yield: 2.2 g (80%).

| | | | | Yield |
|---|---|---|---|---|
| # | Structure | Educt | Method | [%] |
| Z68 | 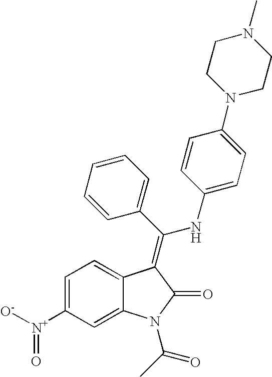 | 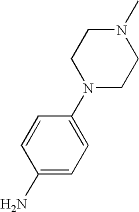 | L | 94 |
| Z69 | 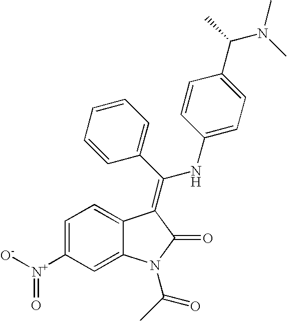 | 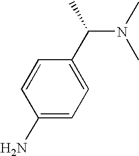 | L | 34 |
| Z70 | 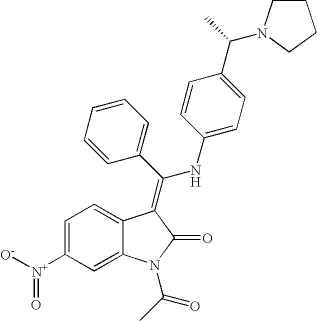 | 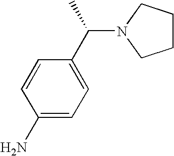 | L | 79 |
| Z71 | 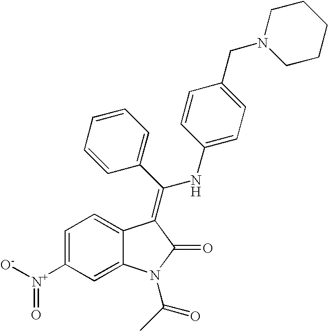 | 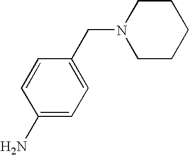 | L | 66 |
| Z72 | 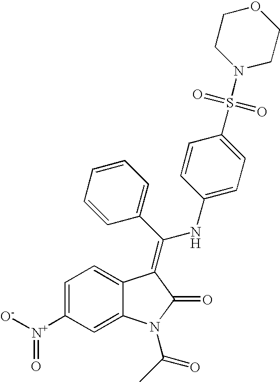 | 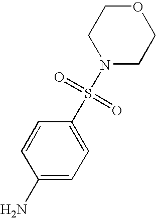 | L | 88 |
| Z73 | 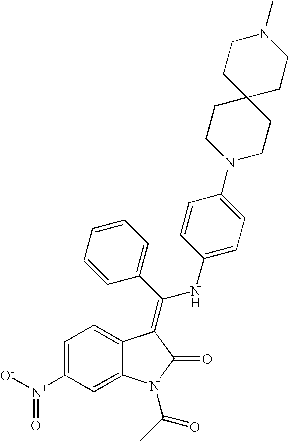 | 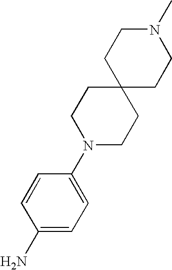 | L | 87 |
| Z74 | 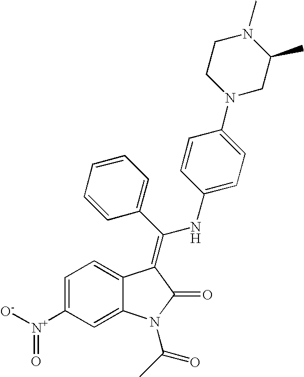 | 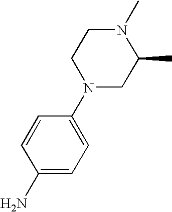 | L | 88 |
| Z75 | 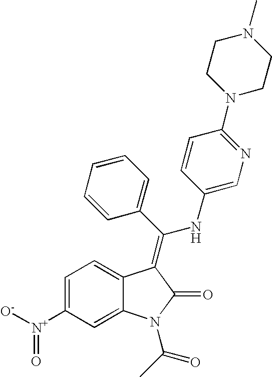 | 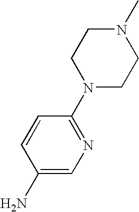 | L | quant. |
| Z76 | 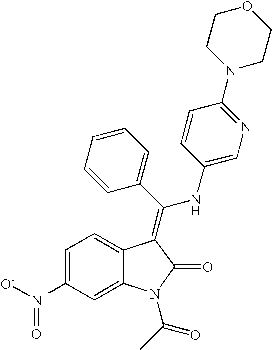 | 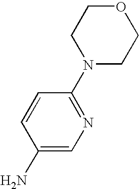 | L | 86 |
| Z77 | 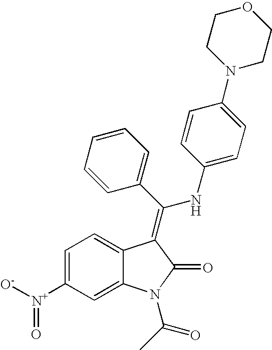 | 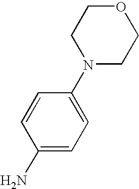 | L | 87 |
| Z78 | 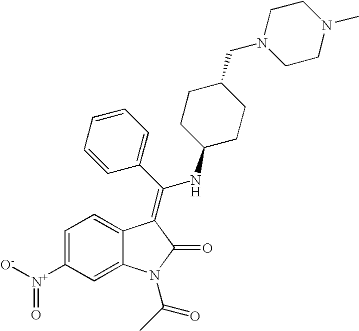 | 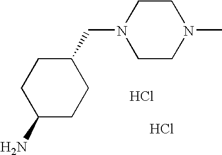 | L | 43 |
| Z79 | 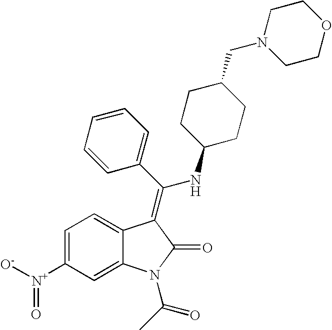 | 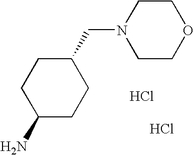 | L | 73 |
| Z80 | 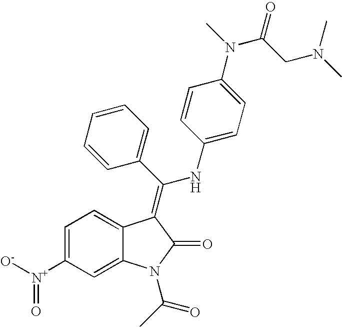 | 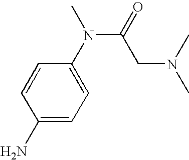 | L | 59 |
| Z81 | 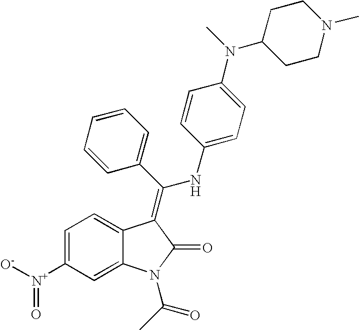 | 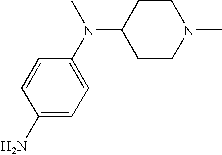 | L | 84 |
| Z82 | 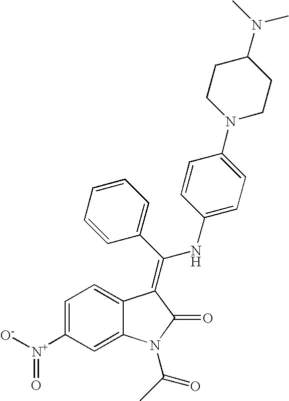 | 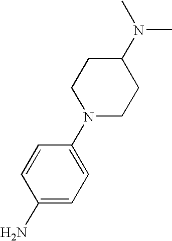 | L | 84 |
| Z83 | 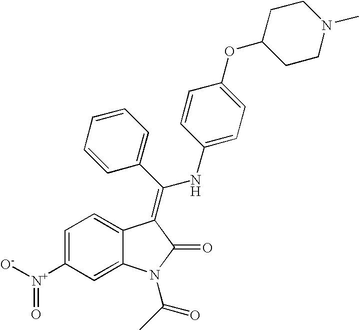 | 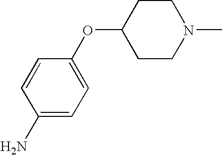 | L | quant. |
| Z84 | 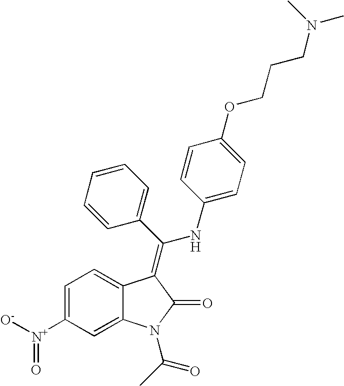 | 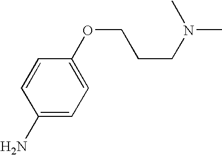 | L | quant. |
| Z85 | 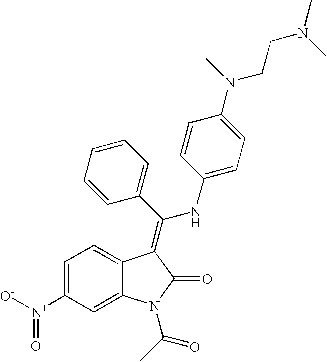 | 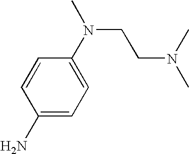 | L | quant. |
| Z86 | 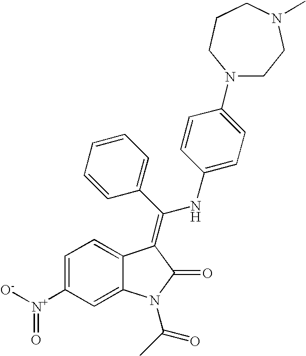 | 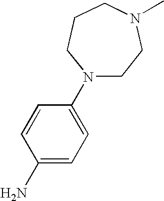 | L | quant. |
| Z87 | 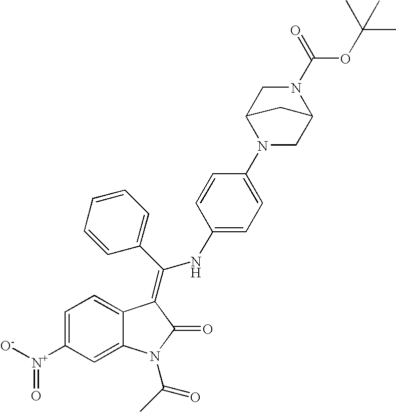 | 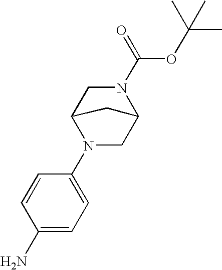 | L | 89 |
| Z88 | 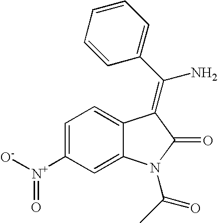 | NH₃ | L | quant. |
| Z89 | 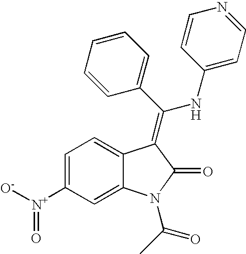 | 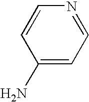 | L | quant. |
| Z90 | 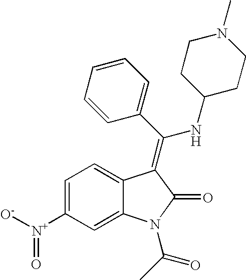 | 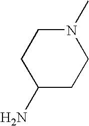 | L | quant. |
| Z91 | 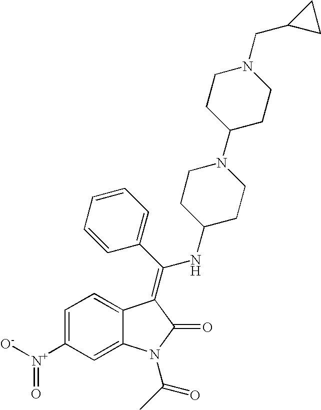 | 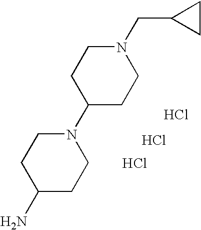 | L | quant. |
| Z92 | 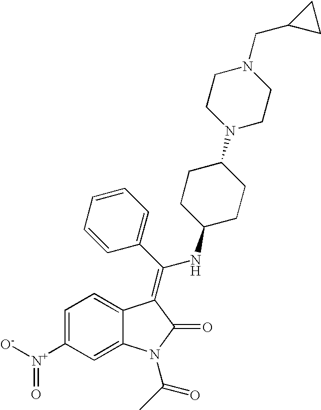 | 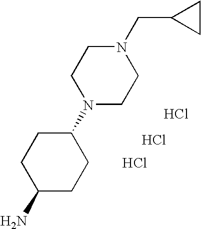 | L | quant. |
| Z93 | 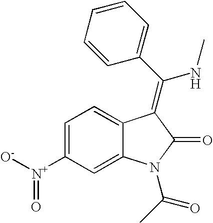 | 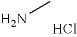 | L | quant. |
| Z94 | 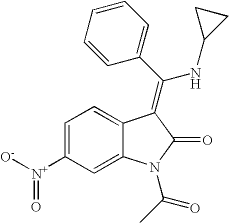 | 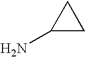 | L | quant. |
| Z95 | 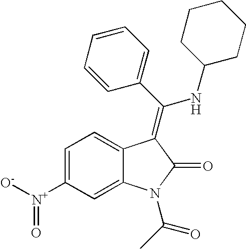 | 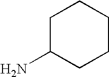 | L | quant. |
| Z96 | 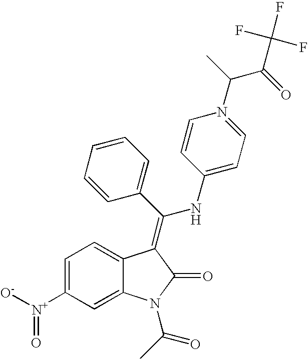 | 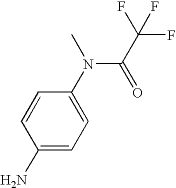 | L | quant. |
| Z97 | 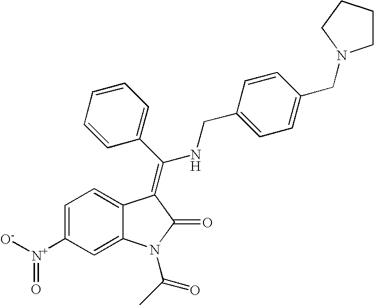 | 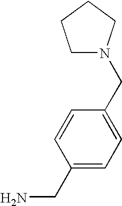 | L | 42 |
| Z98 | 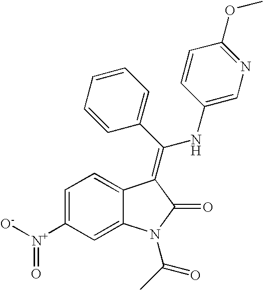 | 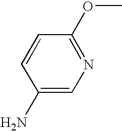 | L | quant. |
| Z99 | 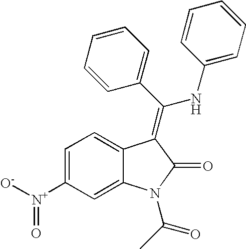 | 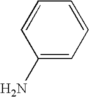 | L | quant. |
| Z100 | 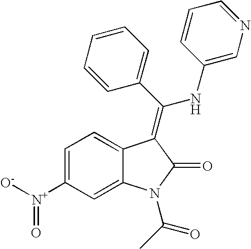 | 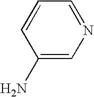 | L | quant. |
| Z101 | 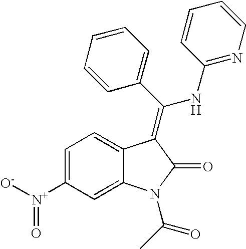 | 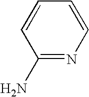 | L | quant. |
| Z102 | 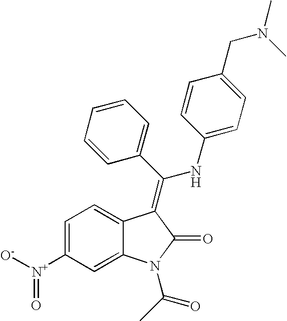 | 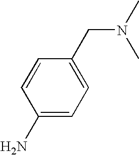 | L | quant. |
| Z103 | 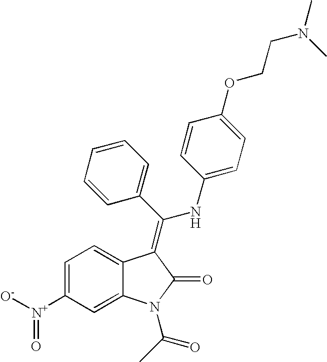 | 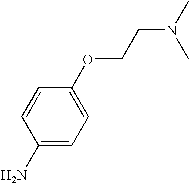 | L | 94 |
| Z104 | 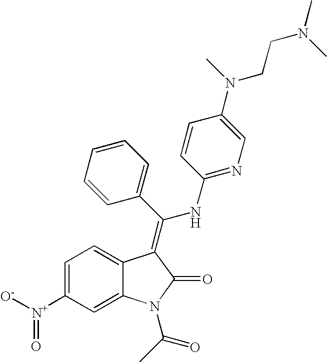 | 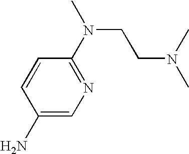 | L | 85 |
| Z105 | 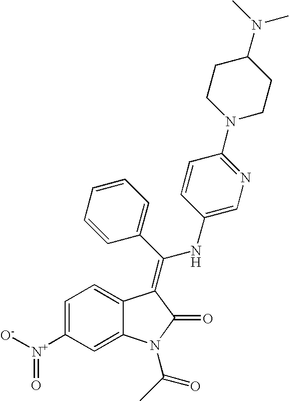 | 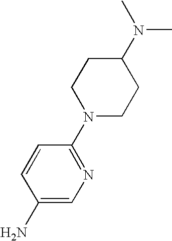 | L | 98 |
| Z106 | 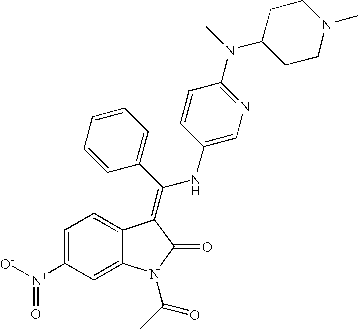 | 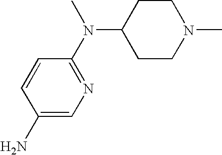 | L | 42 |
| Z107 | 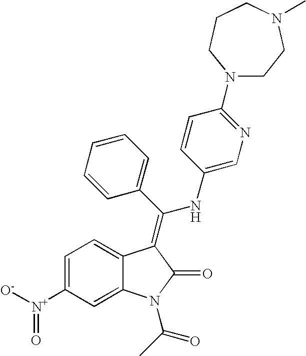 | 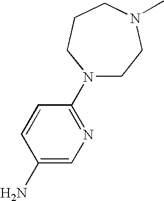 | L | 74 |
| Z108 | 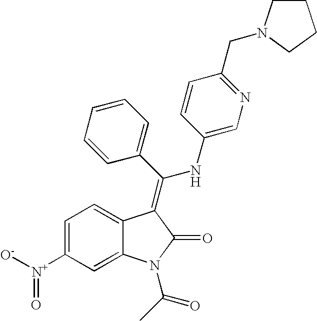 | 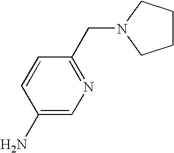 | L | 84 |
| Z109 | 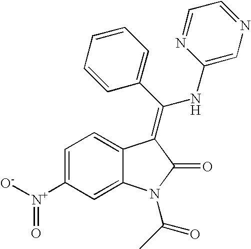 | 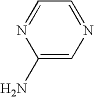 | L | 92 |
| Z110 | 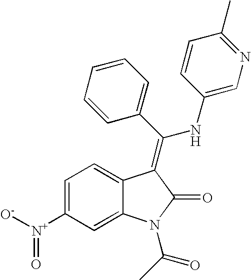 | 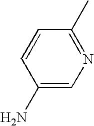 | L | 97 |
| Z111 | 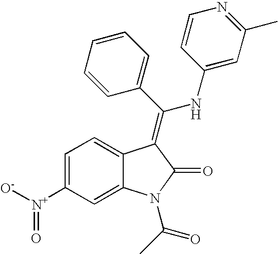 | 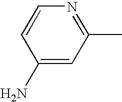 | L | 93 |
| Z112 | 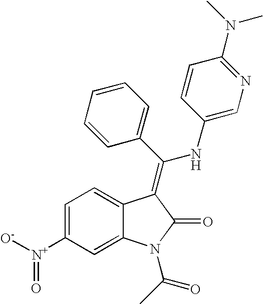 | 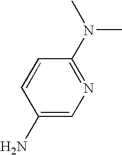 | L | 91 |

### Method M—Reduction of the Nitro Group

(3Z)-1-acetyl-6-nitro-1,3-dihydro-3-[phenyl[[4-(1-pyrrolidinylmethyl)phenyl]amino]-methylene]-2H-indol-2-one (Z113) (1.20 g, 2.49 mmol) in MeOH (25 mL)/CH₂Cl₂ (25 mL) is hydrogenated in the presence of Raney nickel (500 mg) 16 h at RT under a hydrogen pressure of 9 bar. The mixture is filtered and evaporated down. Yield: 1.10 g (98%).

| | | | | Yield |
|---|---|---|---|---|
| # | Structure | Educt | Method | [%] |
| Z114 | 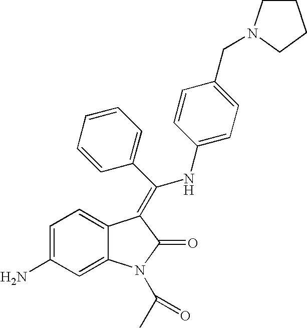 | 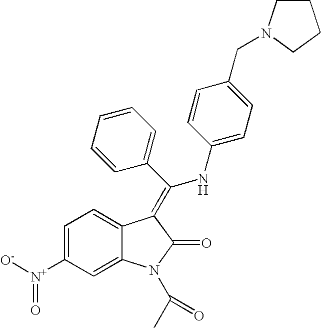 | M | 98 |
| Z115 | 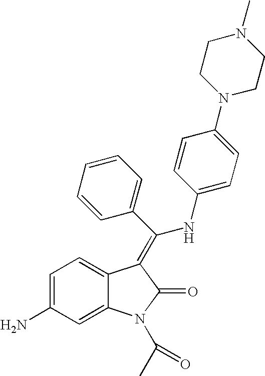 | 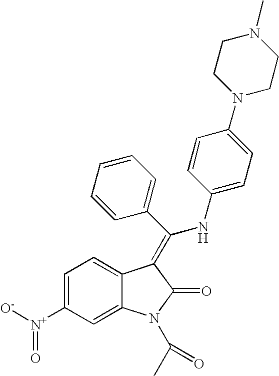 | M | 67 |
| Z116 | 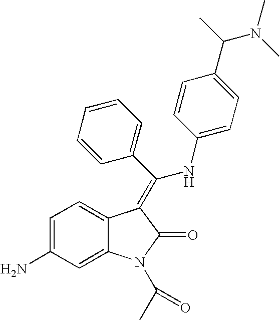 | 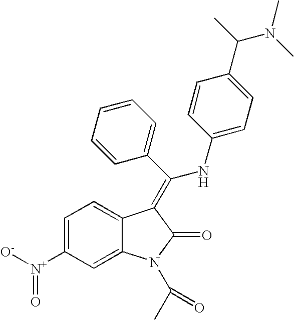 | M | 90 |
| Z117 | 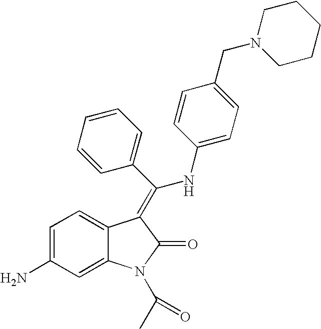 | 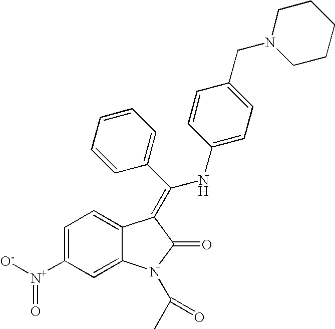 | M | 85 |
| Z118 | 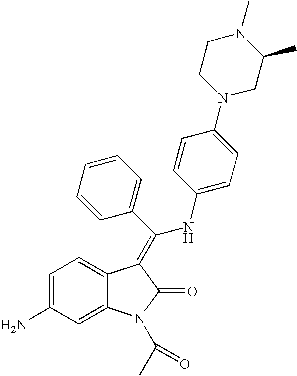 | 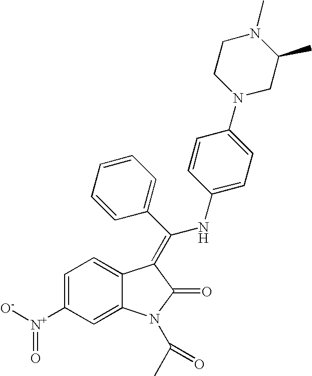 | M | 89 |
| Z119 | 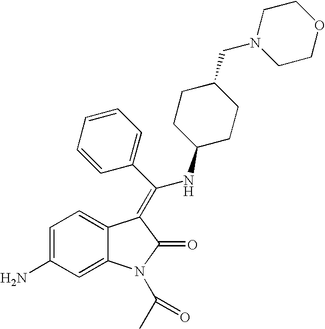 | 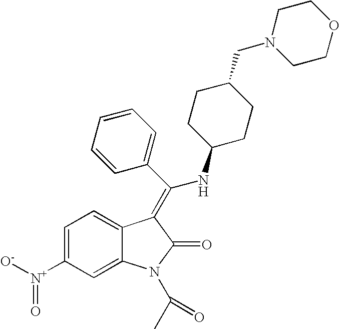 | M | 85 |
| Z120 | 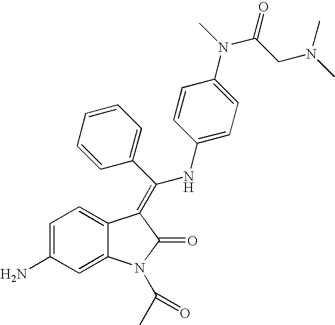 | 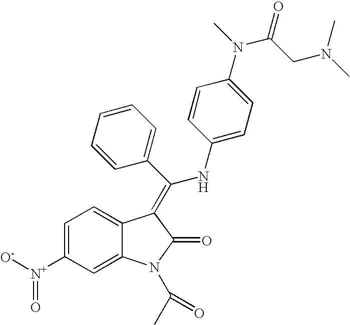 | M | 87 |
| Z121 | 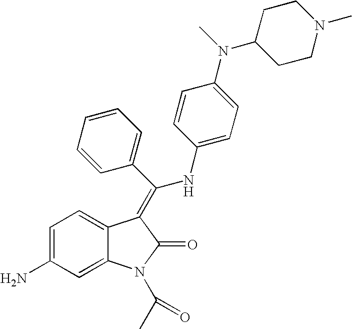 | 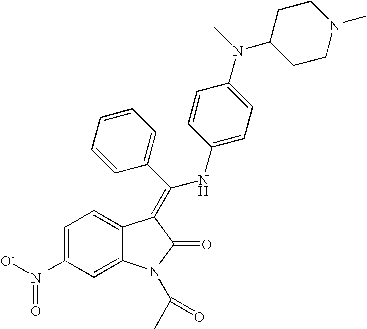 | M | quant. |
| Z122 | 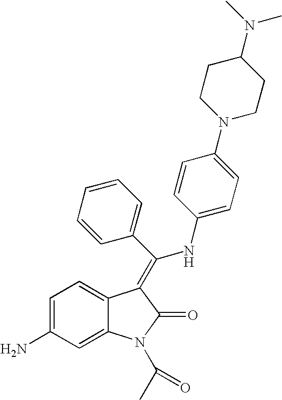 | 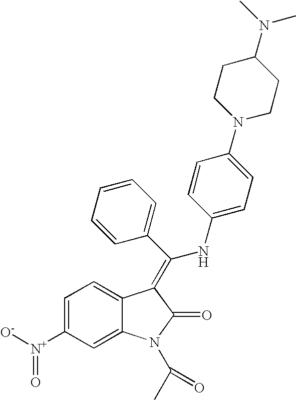 | M | 95 |
| Z123 | 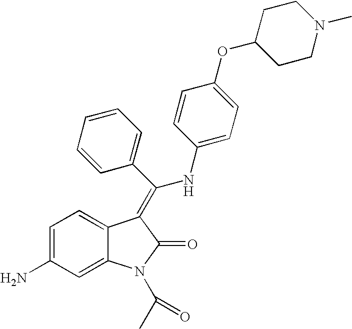 | 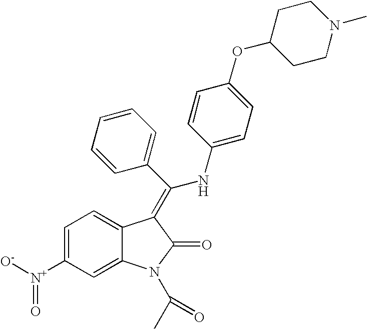 | M | 99 |
| Z124 | 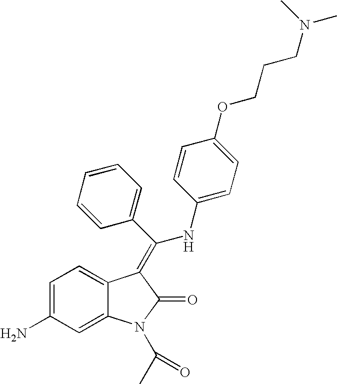 | 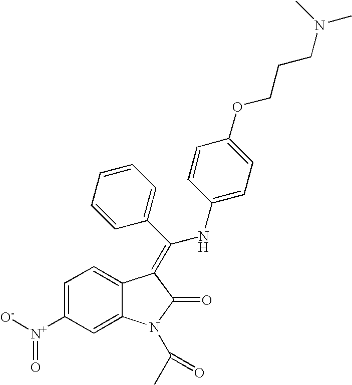 | M | 95 |
| Z125 | 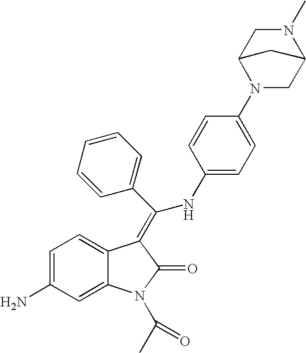 | 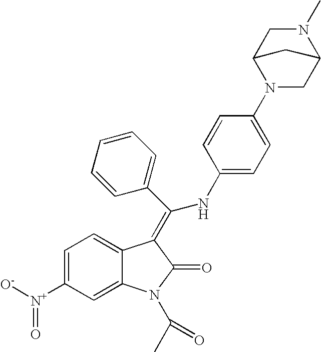 | M | 98 |
| Z126 | 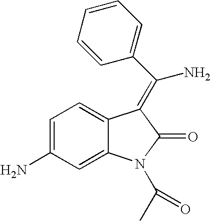 | 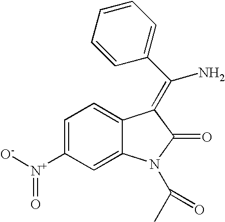 | M | 95 |
| Z127 | 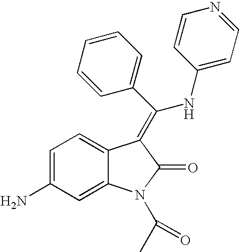 | 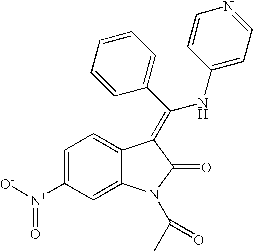 | M | quant. |
| Z128 | 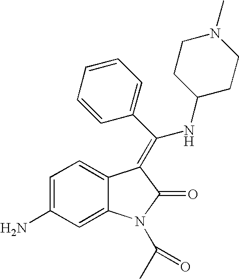 | 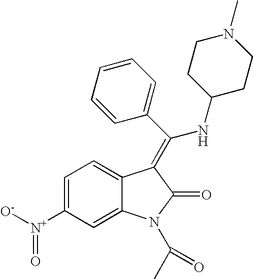 | M | quant. |
| Z129 | 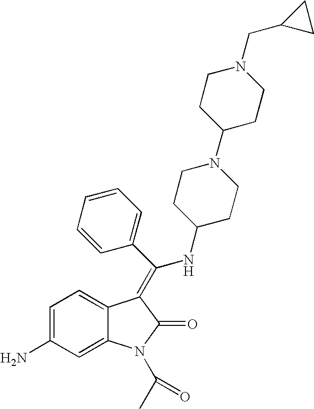 | 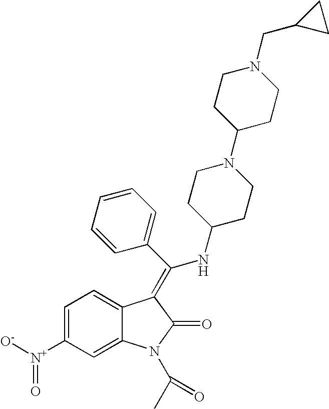 | M | quant. |
| Z130 | 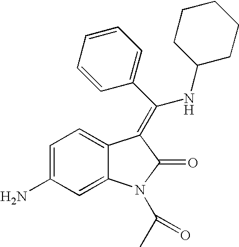 | 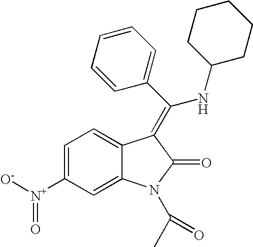 | M | 94 |
| Z131 | 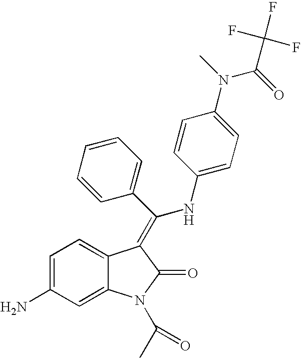 | 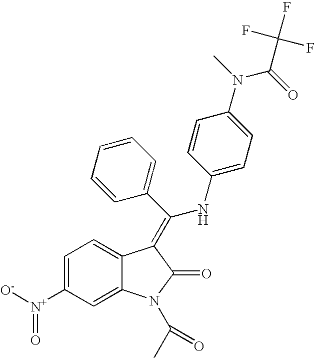 | M | 98 |
| Z132 | 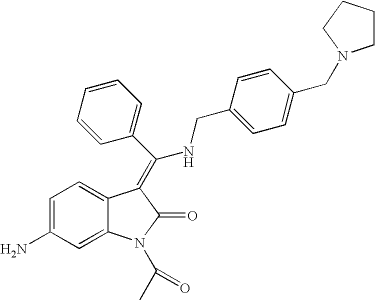 | 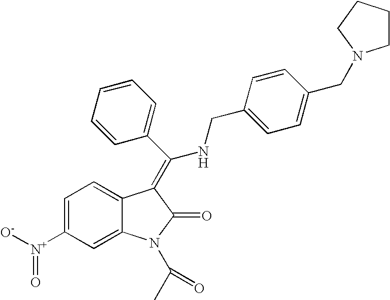 | M | 99 |
| Z133 | 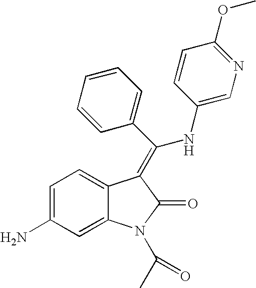 | 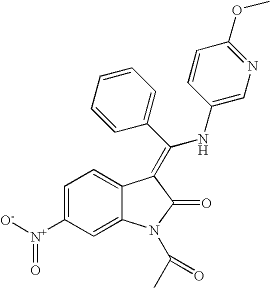 | M | 53. |
| Z134 | 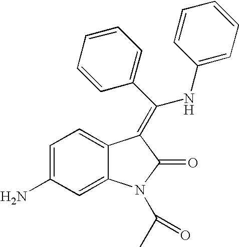 | 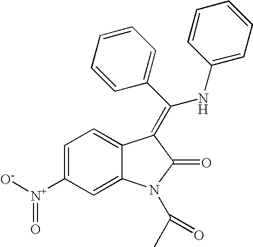 | M | 50 |
| Z135 | 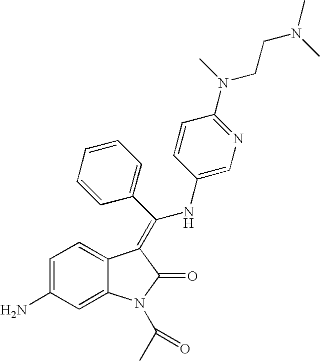 | 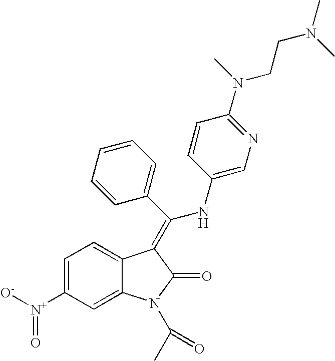 | M | 99 |
| Z136 | 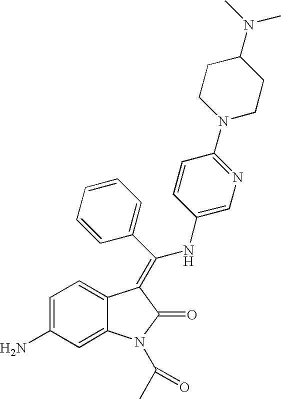 | 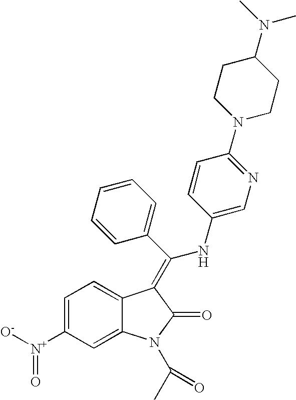 | M | 89 |
| Z137 | 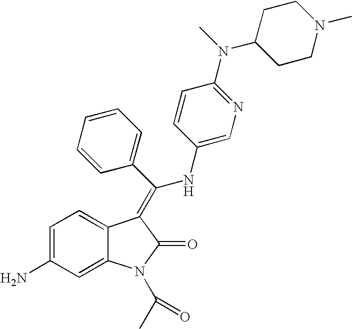 | 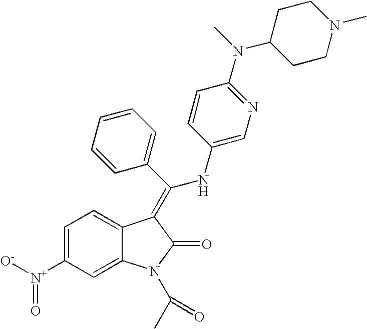 | M | 94 |
| Z138 | 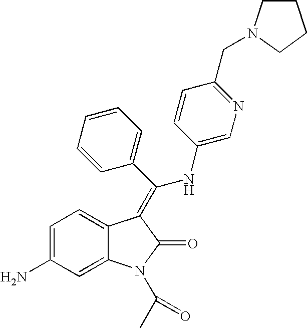 | 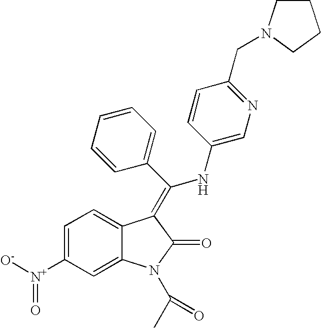 | M | 99 |

### Method P—Cleaving the Acetyl Protective Group

Z115 (1 g, 2.13 mmol) in MeOH (10 mL) is combined with 2 N NaOH (5 mL) and stirred for 1 h at RT. The mixture is evaporated down, mixed with water and the precipitate is filtered off. Yield: 710 mg (78%).

| | | | | Yield |
|---|---|---|---|---|
| # | Structure | Educt | Method | [%] |
| Z139 | 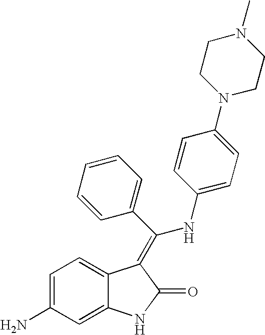 | 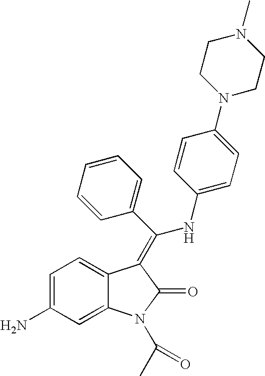 | P | 78 |
| Z140 | 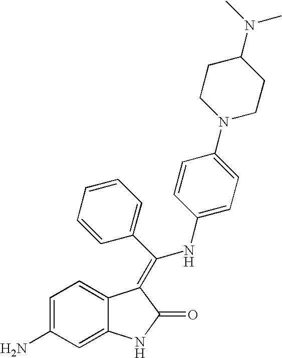 | 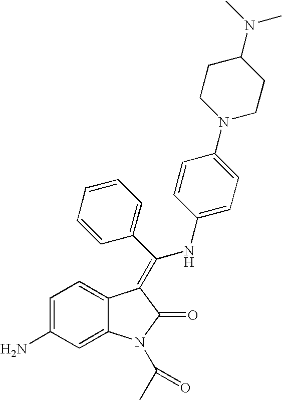 | P | 90 |
| Z141 | 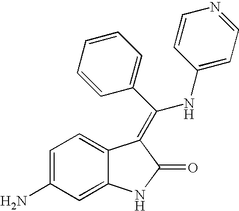 | 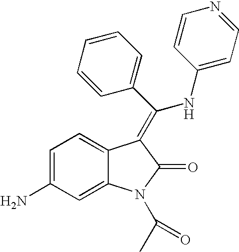 | P | 63 |
| Z142 | 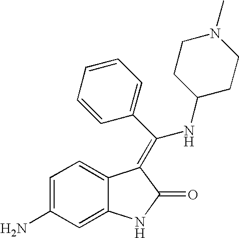 | 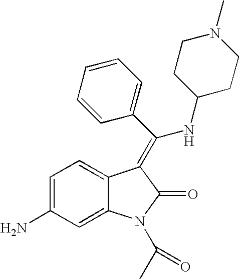 | P | 76 |
| Z143 | 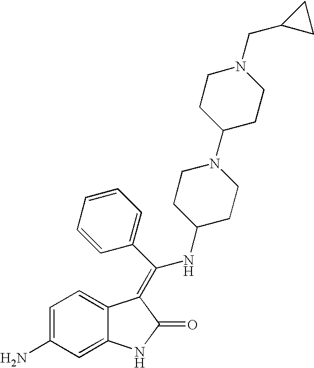 | 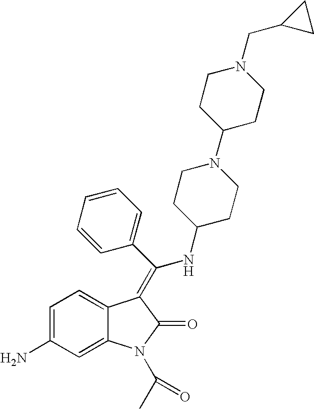 | P | 85 |
| Z144 | 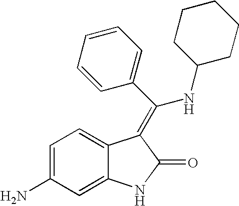 | 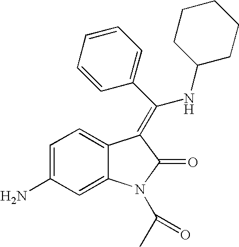 | P | 97 |
| Z145 | 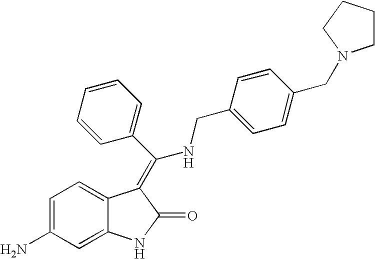 | 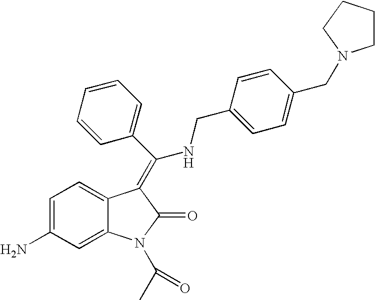 | P | 76 |
| Z146 | 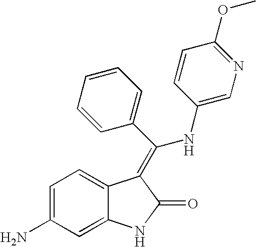 | 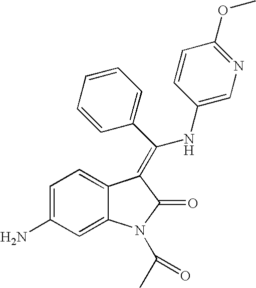 | P | 95 |
| Z147 | 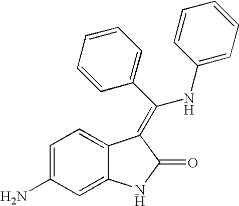 | 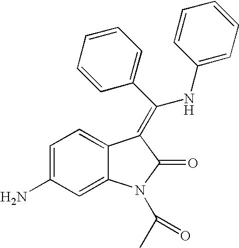 | P | 66 |
| Z148 | 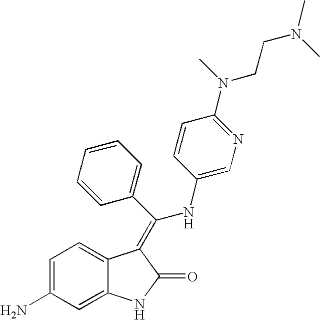 | 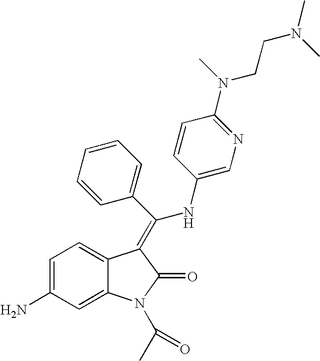 | P | 78 |
| Z149 | 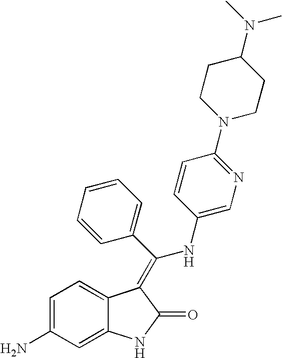 | 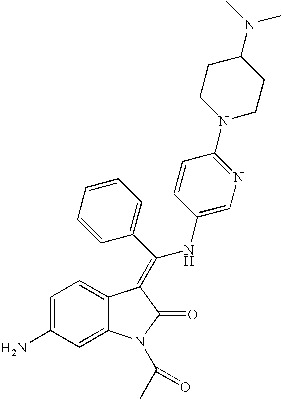 | P | 97 |
| Z150 | 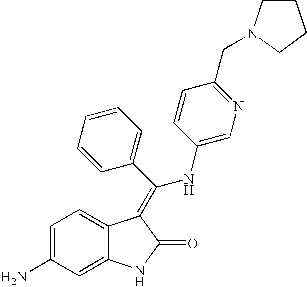 | 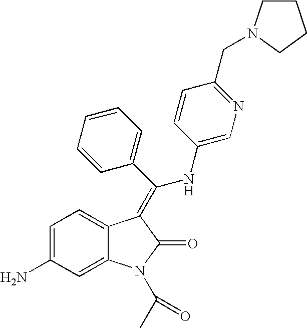 | P | 67 |

### Preparation of Heteroarylmethylidene-Indolinones

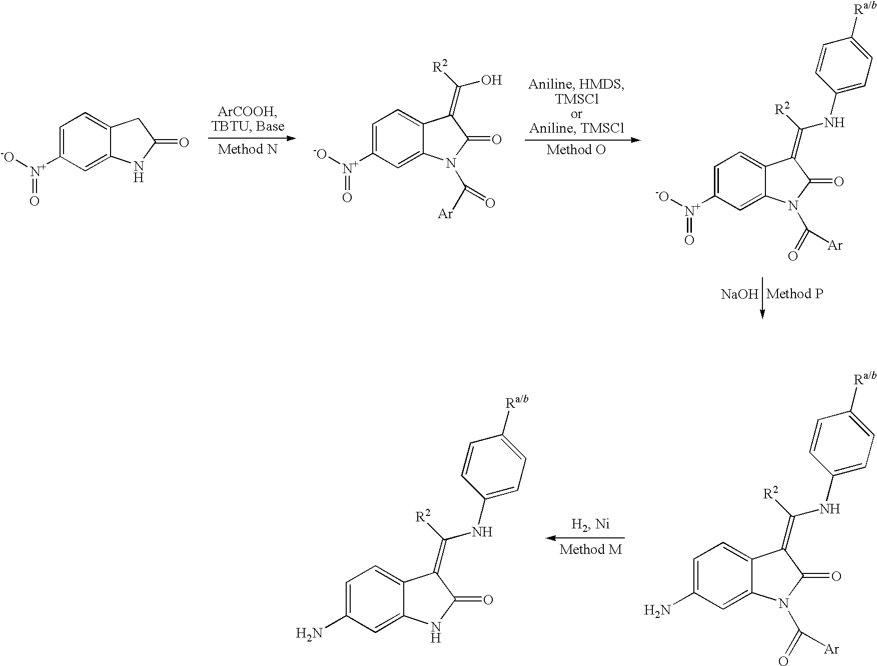

### Method N—Introduction of the Heteroarylmethylidene Fragments

Triethylamine (3.91 mL, 28.0 mmol) and Z4 (1.0 g, 5.61 mmol) are added successively to furan-2-carboxylic acid (1.32 g, 11.79 mmol) and TBTU (3.79 g, 11.79 mmol) in anhydrous DMF (5 mL) and the mixture is stirred for 24 h at RT. The reaction mixture is in poured into 1 N HCl: MeOH=1:1, the precipitate is suction filtered and digested with iPrOH. Yield: 1.60 g (78%).

Alternatively CH₂Cl₂ may be used as solvent. If no crystalline product is obtained, the reaction mixture is worked up by extraction and the residue is optionally chromatographed.

| | | | | Yield |
|---|---|---|---|---|
| # | Structure | Educt | Method | [%] |
| Z151 | 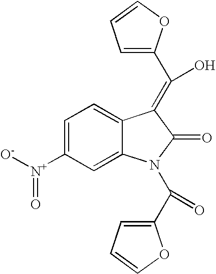 | 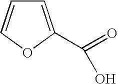 | N | 78 |
| Z152 | 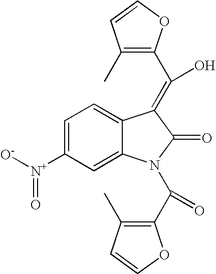 | 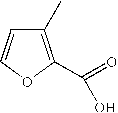 | N | 66 |
| Z153 | 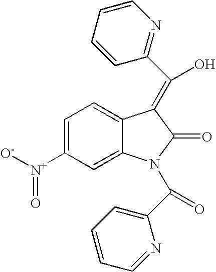 | 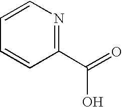 | N | 54 |
| Z154 | 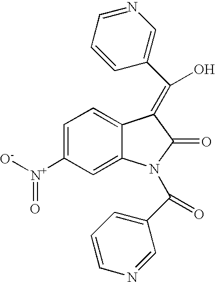 | 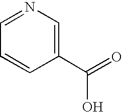 | N | 45 |
| Z155 | 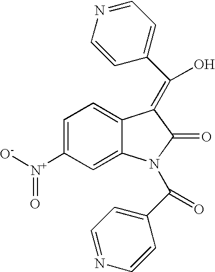 | 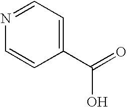 | N | 97 |

Method O—Reaction of the Enols with Aniline Components

Z154 (700 mg, 1.91 mmol), 4-pyrrolidin-1-ylmethylphenylamine (505 mg, 2.87 mmol), TMSCl (1.0 mL, 7.88 mmol) and HMDS (0.81 mL, 3.82 mmol) are stirred in anhydrous THF (8 mL) for 16 h at boiling temperature. The precipitated solid is filtered off, washed with THF and dried. Yield: 900 mg (92%).

Alternatively the reaction may be carried out with aniline components in the presence of 3 equivalents of TMSCl in THF in the microwave (160° C., 15 min).

In the reaction deacetylated product is obtained at the indolinone nitrogen and may optionally occur as the main product and be reacted further. The yields are given as the total of main product and by-product.

| | | | | Yield |
|---|---|---|---|---|
| # | Structure | Educt | Method | [%] |
| Z156 | 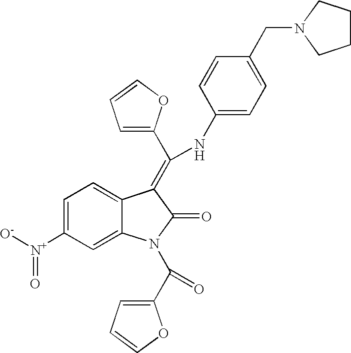 | 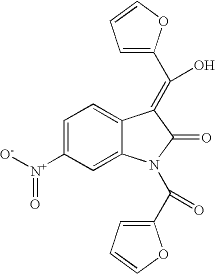 | O | 92 |
| Z157 | 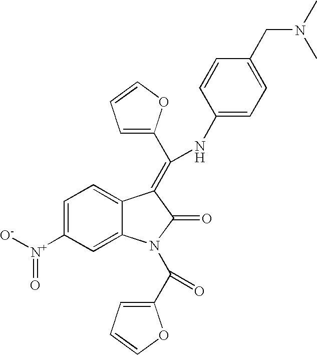 | 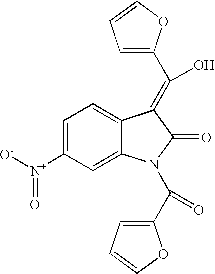 | O | 80 |
| Z158 | 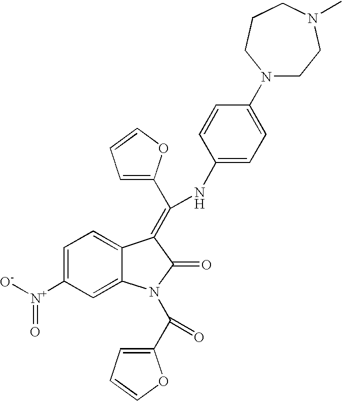 | 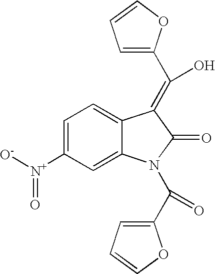 | O | 55 |
| Z159 | 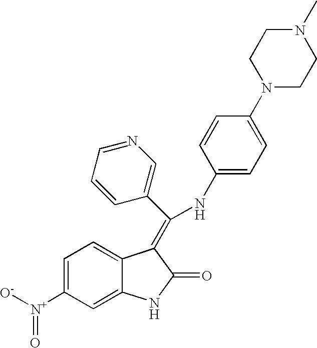 | 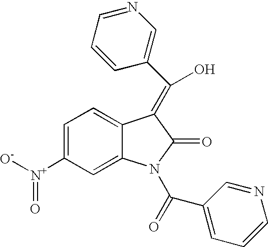 | O | 26 |
| Z160 | 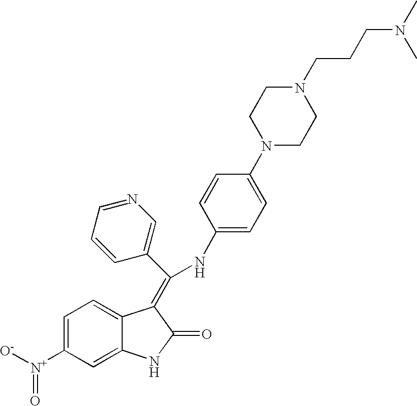 | 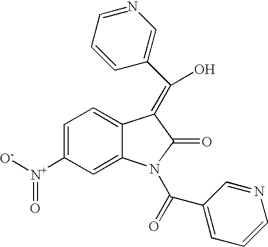 | O | 77 |
| Z161 | 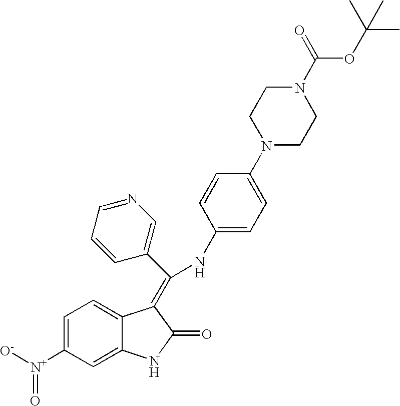 | 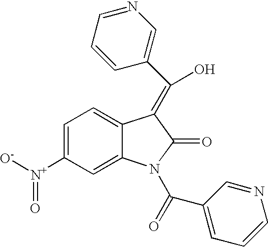 | O | 43 |
| Z162 | 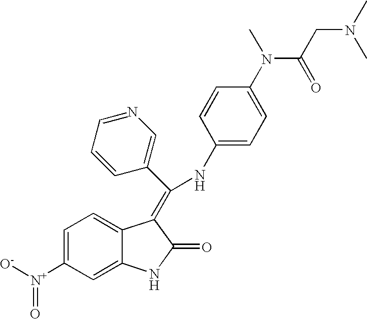 | 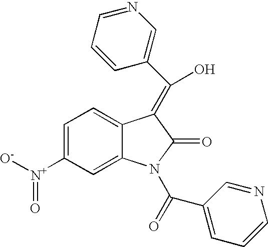 | O | 87 |
| Z163 | 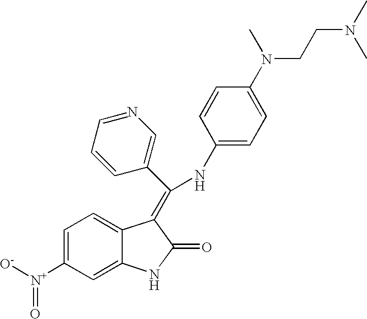 | 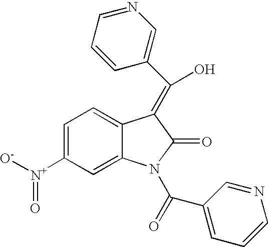 | O | 74 |
| Z164 | 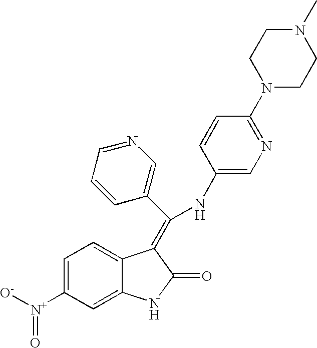 | 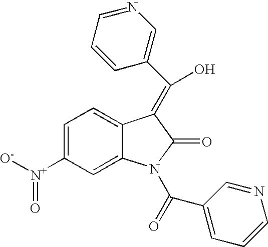 | O | 28 |
| Z165 | 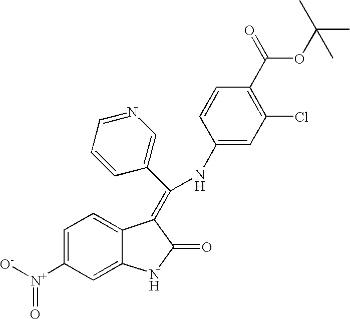 | 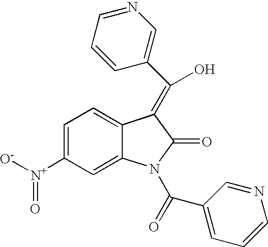 | O | 45 |
| Z166 | 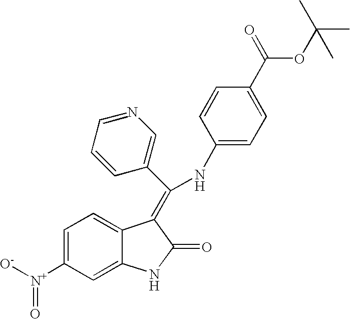 | 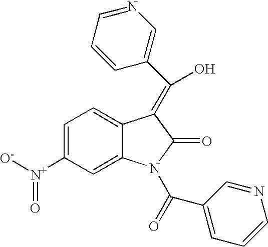 | O | 53 |
| Z167 | 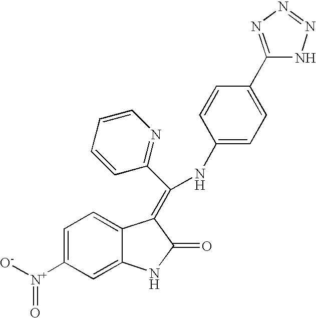 | 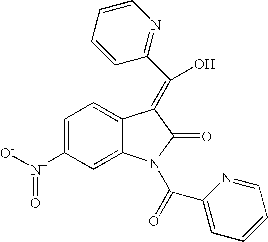 | O | quant. |
| Z168 | 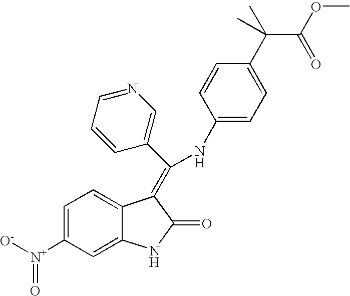 | 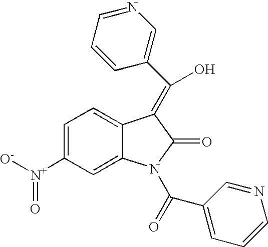 | O | 98 |
| Z169 | 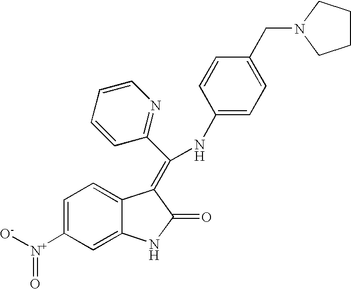 | 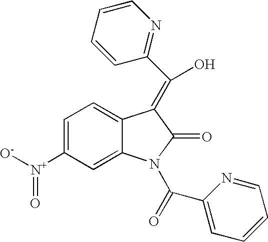 | O | 97 |
| Z170 | 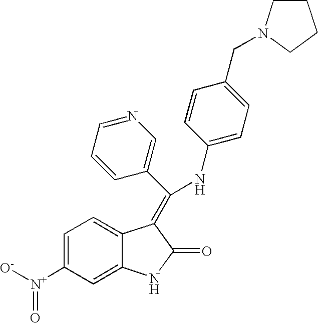 | 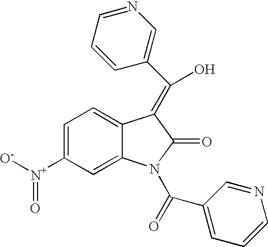 | O | 47 |
| Z171 | 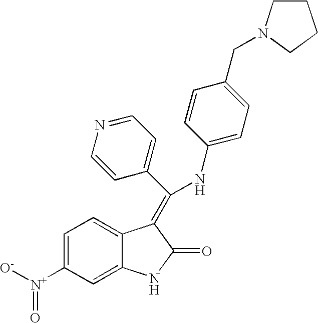 | 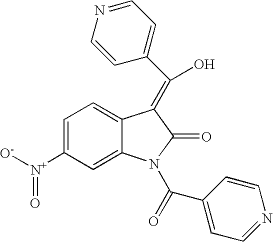 | O | 45 |
| Z172 | 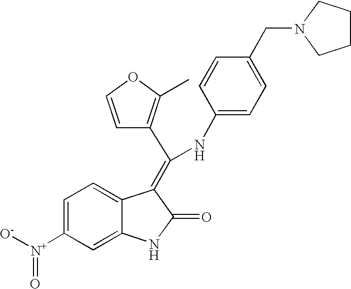 | 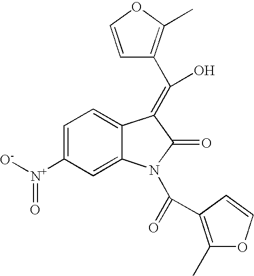 | O | 23 |

The reduction of the nitro group is carried out according to Method M.

| | | | | Yield |
|---|---|---|---|---|
| # | Structure | Educt | Method | [%] |
| Z173 | 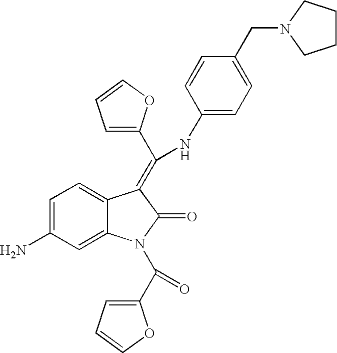 | 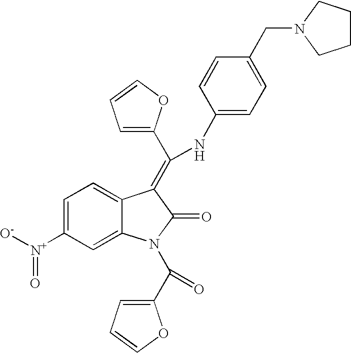 | M | 49 |
| Z174 | 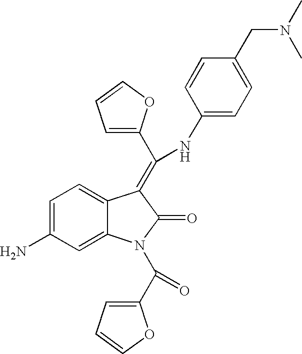 | 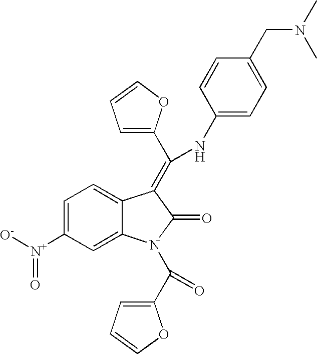 | M | 86 |
| Z175 | 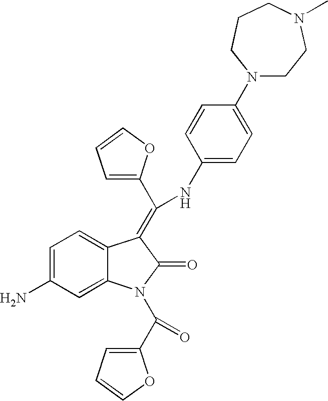 | 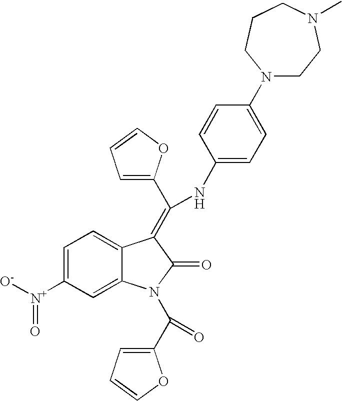 | M | 76 |
| Z176 | 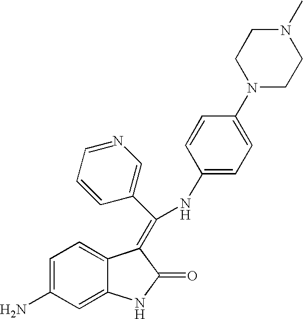 | 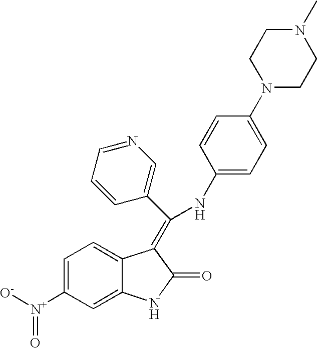 | M | 89 |
| Z177 | 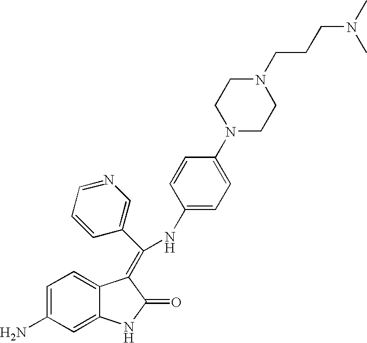 | 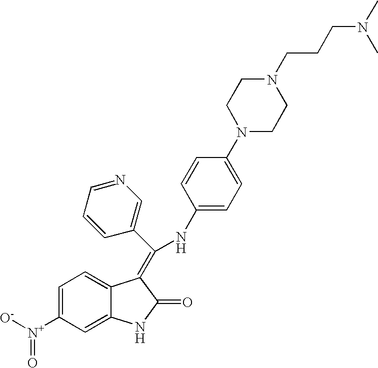 | M | 93 |
| Z178 | 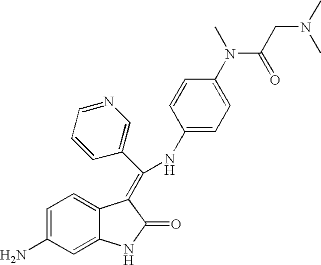 | 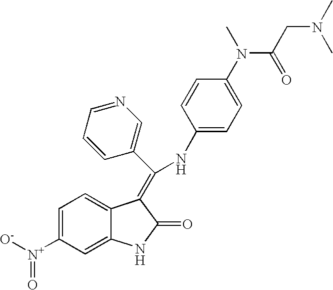 | M | 81 |
| Z179 | 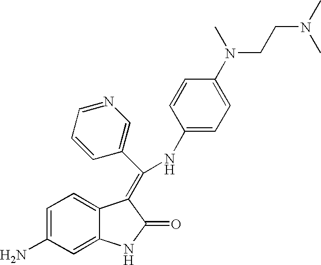 | 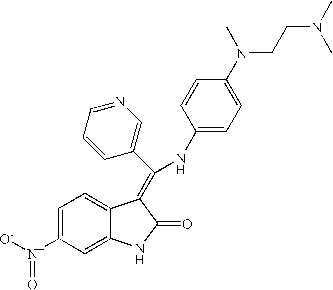 | M | 81 |
| Z180 | 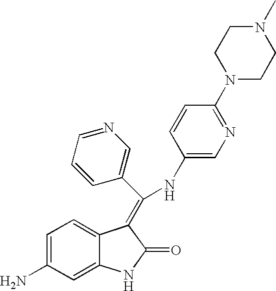 | 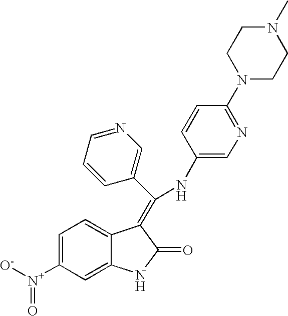 | M | 81 |
| Z181 | 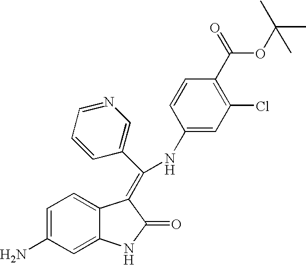 | 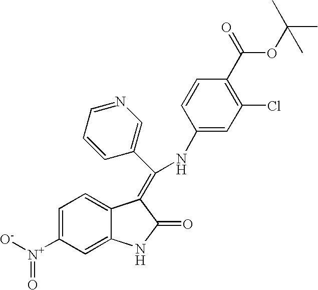 | M | 93 |
| Z182 | 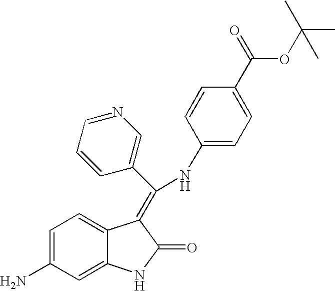 | 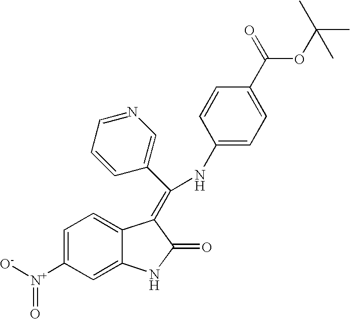 | M | 99 |
| Z183 | 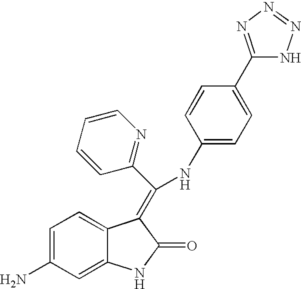 | 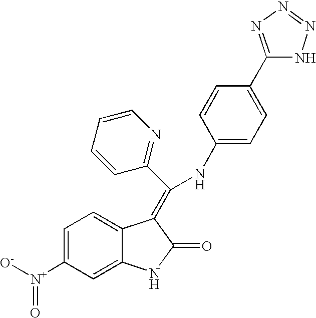 | M | 99 |
| Z184 | 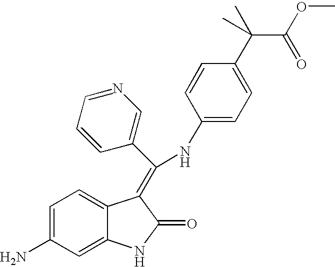 | 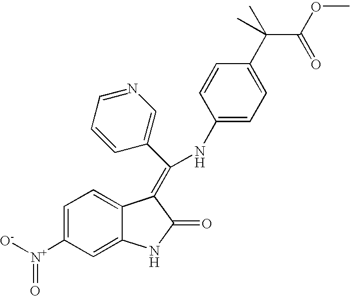 | M | 99 |
| Z185 | 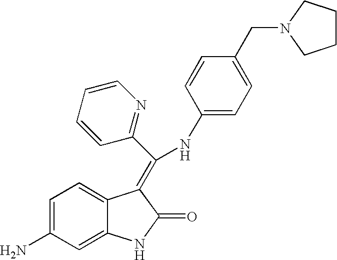 | 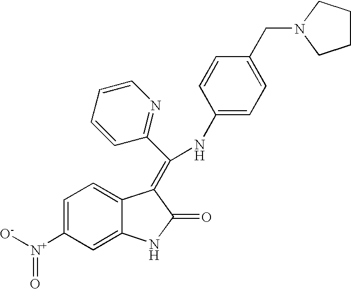 | M | 96 |
| Z186 | 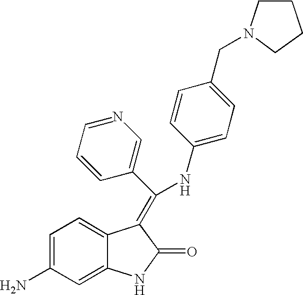 | 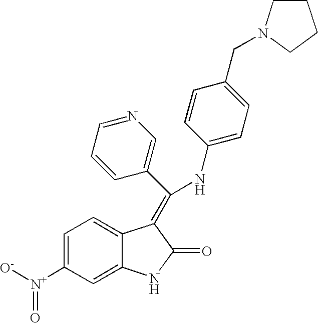 | M | 85 |
| Z187 | 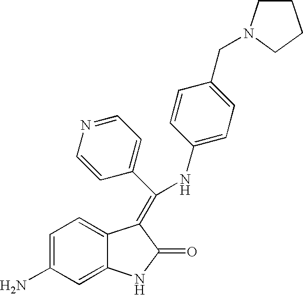 | 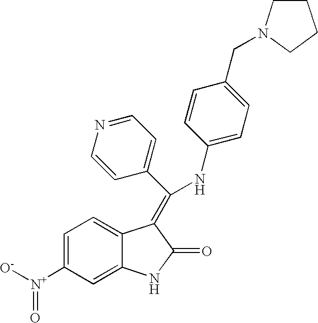 | M | 93 |
| Z188 | 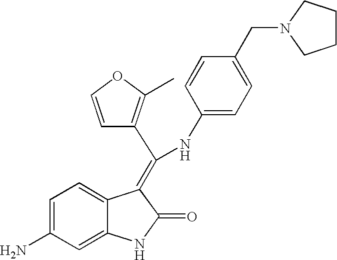 | 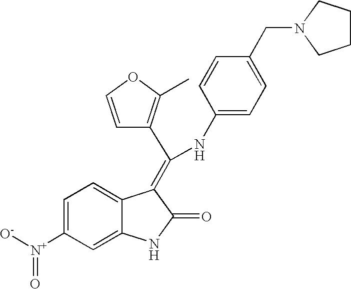 | M | 98 |

The cleaving of the amide protective group at the indolinone-nitrogen is carried out according to Method P using NaOH or conc. ammonia.

| | | | | Yield |
|---|---|---|---|---|
| # | Structure | Educt | Method | [%] |
| Z189 | 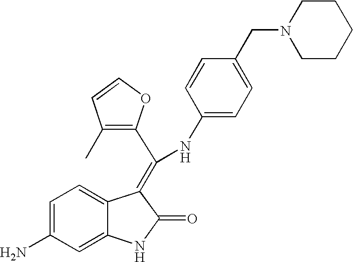 | 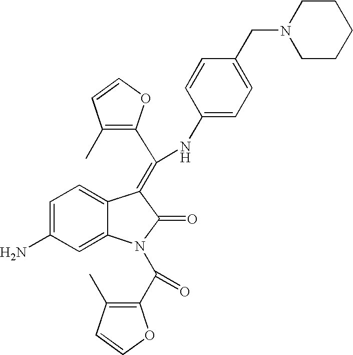 | P | 57 |

### Method Q—Amide Formation

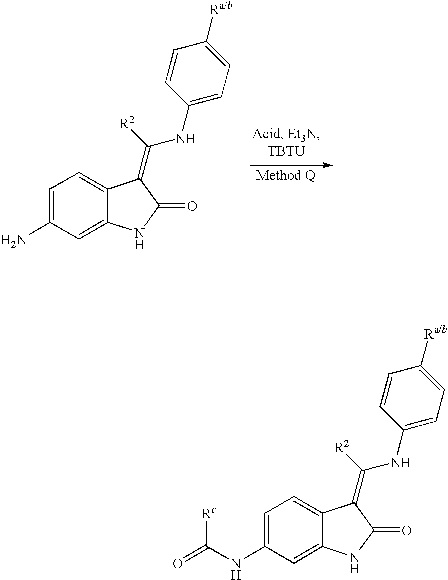

Triethylamine (1.2 equiv) is added to a solution of the carboxylic acid (1 equiv) and TBTU (1.2 equiv) in anhydrous DMSO or NMP (5 μL/1 mg aniline) and shaken for 5 min at ambient temperature. The aniline (1 equiv) is added to anhydrous DMSO or NMP (5 μL/1 mg aniline) and shaken for 30 min at RT. The reaction mixture is filtered and purified by preparative HPLC.

TABLE 1
Phenylmethylidene compounds

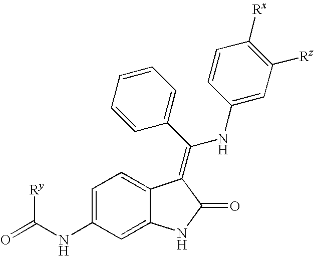

| | | | | tᵣₑₜ | | UVₘₐₓ | HPLC- |
|---|---|---|---|---|---|---|---|
| Ex. | R^{y} | R^{x} | R^{z} | [min] | [M + H]⁺ | [nM] | Method |
| 1 | 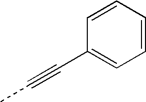 | 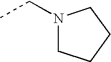 | H | 1.75 | 539.5 | 259 | A |
| 2 | 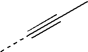 | 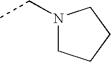 | H | 1.54 | 477.5 | 296 | A |
| 3 | 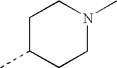 | 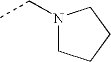 | H | 0.12 | 536.3 | 286 | A |
| 4 | 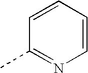 | 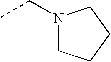 | H | 1.67 | 516.5 | 397 | A |
| 5 | 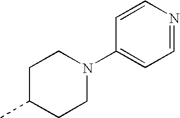 | 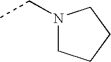 | H | 1.53 | 300.5 (half mass) | 286 | A |
| 6 | 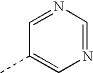 | 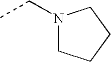 | H | 0.12 | 517.3 | 287 | A |
| 7 | 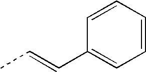 | 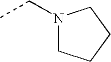 | H | 1.74 | 541.5 | 283 | A |
| 8 | 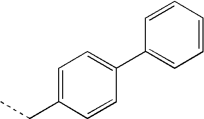 | 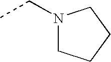 | H | 1.84 | 605.5 | 257 | A |
| 9 | 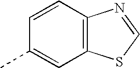 | 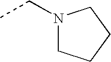 | H | 1.62 | 572.3 | 263 | A |
| 10 | 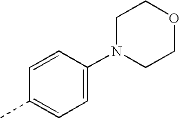 | 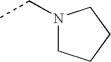 | H | 1.66 | 600.3 | 295 | A |
| 11 | 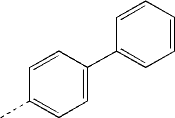 | 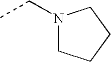 | H | 1.86 | 591.5 | 277 | A |
| 12 | 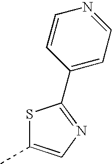 | 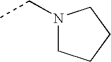 | H | 1.62 | 599.2 | 289 | A |
| 13 | 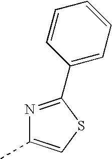 | 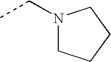 | H | 1.86 | 598.3 | 291 | A |
| 14 | 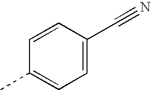 | 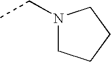 | H | 1.64 | 540.3 | 396 | A |
| 15 | 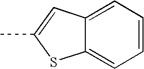 | 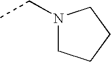 | H | 1.81 | 571.3 | 288 | A |
| 16 | 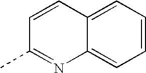 | 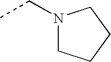 | H | 1.83 | 566.5 | 286 | A |
| 17 | 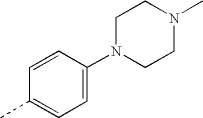 | 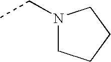 | H | 1.38 | 613.3 | 294 | A |
| 18 | 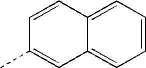 | 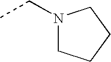 | H | 1.80 | 565.3 | 285 | A |
| 19 | 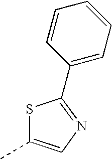 | 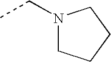 | H | 1.80 | 598.3 | 293 | A |
| 20 | 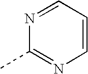 | 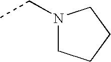 | H | 1.50 | 517.3 | 291 | A |
| 21 | 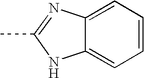 | 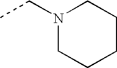 | H | 1.58 | 569.3 | 289 | A |
| 22 | 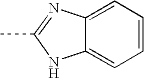 | 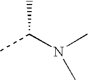 | H | 1.54 | 543.3 | 288 | A |
| 23 | 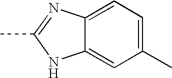 | 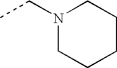 | H | 1.64 | 583.3 | 290 | A |
| 24 | 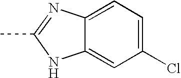 | 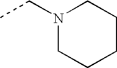 | H | 1.69 | 604.3 | 289 | A |
| 25 | 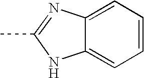 | 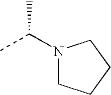 | H | 1.73 | 569.3 | 288 | A |
| 26 | 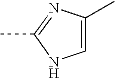 | 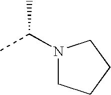 | H | 1.56 | 462.3 | 271 | A |
| 27 | 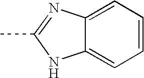 | 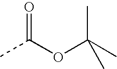 | H | 2.66 | 572.3 | 293 | A |
| 28 | 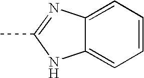 | 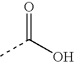 | H | 1.37 | 516.3 | 290 | B |
| 29 | 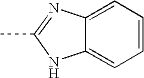 | 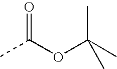 | F | 2.66 | 590.3 | 292 | A |
| 30 | 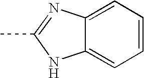 | 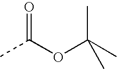 | Cl | 2.71 | 607.3 | 291 | A |
| 31 | 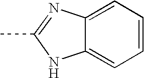 | 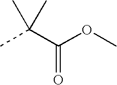 | H | 2.44 | 572.3 | 291 | A |
| 32 | 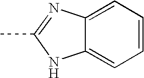 | 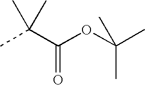 | H | 2.72 | 614.3 | 288 | A |
| 33 | 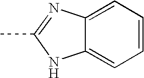 | 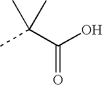 | H | 2.26 | 558.3 | 288 | A |
| 34 | 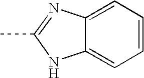 | 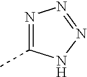 | H | 2.09 | 540.3 | 292 | A |
| 35 | 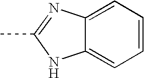 | 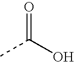 | F | 2.15 | 534.3 | 293 | A |
| 36 | 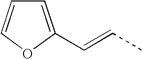 | 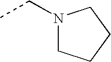 | H | 1.66 | 531.2 | 297 | A |
| 37 | 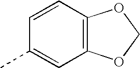 | 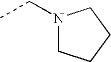 | H | 166 | 559.3 | 294 | A |
| 38 | 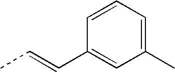 | 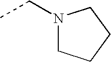 | H | 1.79 | 555.5 | 285 | A |
| 39 | 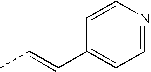 | 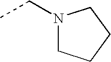 | H | 1.42 | 542.3 | 280 | A |
| 40 | 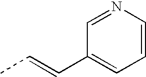 | 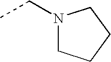 | H | 0.12 | 542.3 | 283 | A |
| 41 | 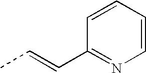 | 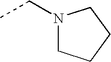 | H | 1.56 | 542.5 | 289 | A |
| 42 | 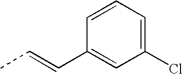 | 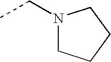 | H | 1.68/ 1.81 (cis/ trans) | 575.2 | 281 | A |
| 43 | 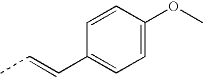 | 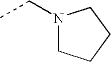 | H | 1.75 | 571.5 | 294 | A |
| 44 | 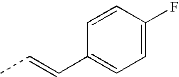 | 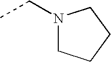 | H | 1.75 | 559.3 | 284 | A |
| 45 | 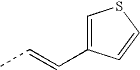 | 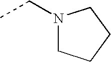 | H | 1.69 | 547.2 | 289 | A |
| 46 | 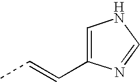 | 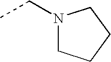 | H | 0.12 | 531.5 | 273 | A |
| 47 | 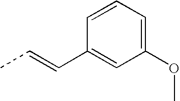 | 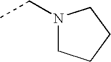 | H | 1.74 | 571.3 | 284 | A |
| 48 | 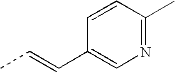 | 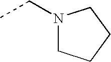 | H | 0.12 | 556.3 | 283 | A |
| 49 | 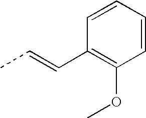 | 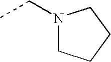 | H | 1.75 | 571.5 | 284 | A |
| 50 | 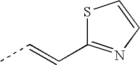 | 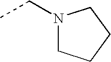 | H | 1.61 | 548.3 | 293 | A |
| 51 | 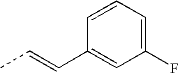 | 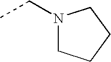 | H | 1.76 | 559.3 | 281 | A |
| 52 | 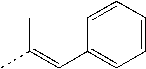 | 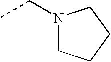 | H | 1.80 | 555.3 | 274 | A |
| 53 | 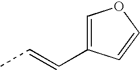 | 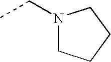 | H | 1.67 | 531.2 | 285 | A |
| 54 | 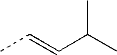 | 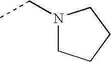 | H | 1.71 | 507.5 | 292 | A |
| 55 | 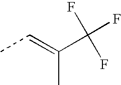 | 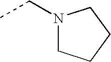 | H | 1.74 | 547.2 | 290 | A |
| 56 | 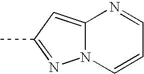 | 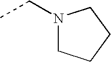 | H | 1.58 | 556.5 | 288 | A |
| 57 | 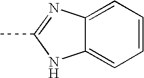 | 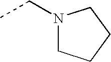 | H | 1.69 | 555.3 | 289 | A |
| 58 | 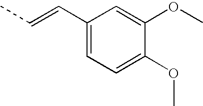 | 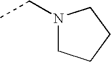 | H | 1.69 | 601.3 | 334 | A |
| 59 | 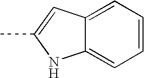 | 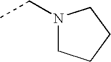 | H | 1.75 | 554.2 | 314 | A |
| 60 | 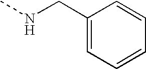 | 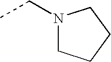 | H | 1.63 | 544.5 | 396 | A |
| 61 | 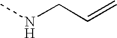 | 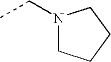 | H | 1.53 | 494.5 | 395 | A |
| 62 | 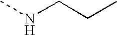 | 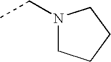 | H | 1.54 | 496.5 | 393 | A |
| 63 | 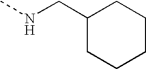 | 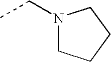 | H | 1.75 | 550.5 | 396 | A |
| 64 | 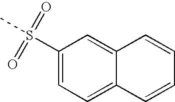 | 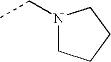 | H | 1.73 | 601.3 | 389 | A |
| 65 | 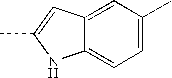 | 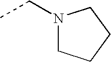 | H | 1.81 | 498.5 | 318 | A |
| 66 | 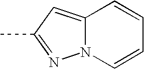 | 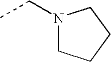 | H | 1.69 | 555.3 | 396 | A |
| 67 | 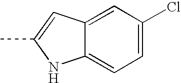 | 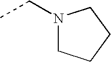 | H | 1.82 | 589.3 | 398 | A |
| 68 | 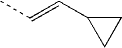 | 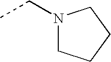 | H | 1.65 | 505.5 | 398 | A |
| 69 | 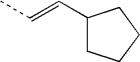 | 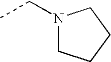 | H | 1.77 | 533.5 | 294 | A |
| 70 | 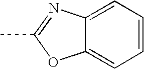 | 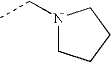 | H | 1.70 | 556.5 | 282 | A |
| 71 | 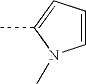 | 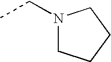 | H | 1.66 | 518.3 | 396 | A |
| 72 | 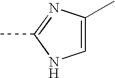 | 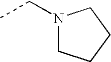 | H | 1.51 | 519.3 | 398 | A |
| 73 | 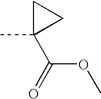 | 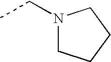 | H | 1.67 | 537.5 | 392 | A |
| 74 | 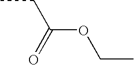 | 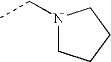 | H | 1.53 | 525.5 | 391 | A |
| 75 | 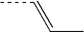 | 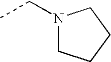 | H | 1.56 | 479.5 | 396 | A |
| 76 | 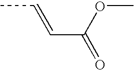 | 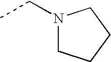 | H | 1.65 | 537.3 | 284 | A |
| 77 | 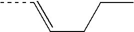 | 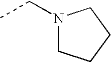 | H | 1.69 | 507.5 | 396 | A |
| 78 | 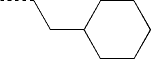 | 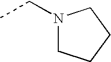 | H | 1.86 | 549.8 | 287 | A |
| 79 | 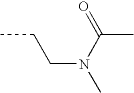 | 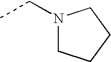 | H | 0.12 | 538.5 | 390 | A |
| 80 | 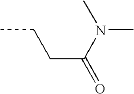 | 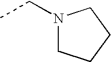 | H | 0.12 | 538.5 | 390 | A |
| 81 | 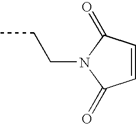 | 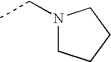 | H | 1.51 | 562.5 | 391 | A |
| 82 | 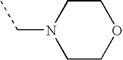 | 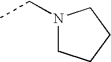 | H | 0.12 | 538.3 | 388 | A |
| 83 | 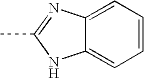 | 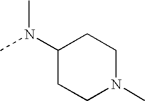 | H | 1.73 | 598.3 | 289 | A |
| 84 | 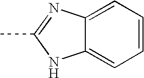 | 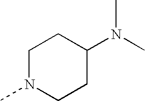 | H | 1.72 | 598.5 | 290 | A |
| 85 | 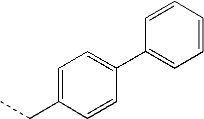 | 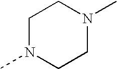 | H | 1.84 | 620.3 | 393 | A |
| 86 | 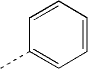 | 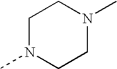 | H | 1.63 | 530.3 | 390 | A |
| 87 | 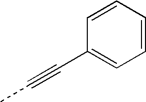 | 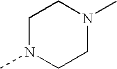 | H | 1.73 | 554.3 | 396 | A |
| 88 | 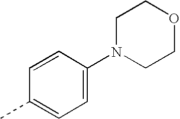 | 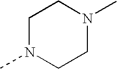 | H | 1.63 | 615.5 | 396 | A |
| 89 | 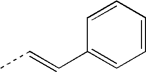 | 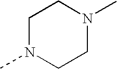 | H | 1.73 | 556.3 | 285 | A |
| 90 | 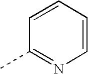 | 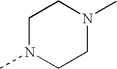 | H | 1.66 | 531.5 | 397 | A |
| 91 | 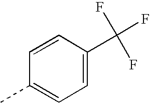 | 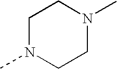 | H | 1.78 | 598.3 | 393 | A |
| 92 | 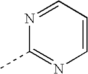 | 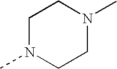 | H | 1.50 | 532.3 | 397 | A |
| 93 | 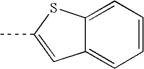 | 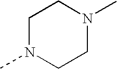 | H | 1.79 | 586.3 | 287 | A |
| 94 | 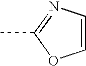 | 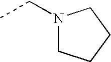 | H | 1.51 | 506.5 | 398 | A |
| 95 | 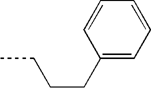 | 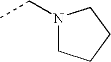 | H | 1.73 | 557.3 | 392 | A |
| 96 | 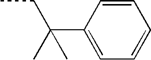 | 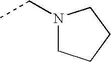 | H | 1.80 | 571.5 | 393 | A |
| 97 | 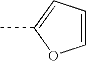 | 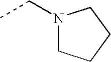 | H | 1.57 | 505.5 | 394 | A |
| 98 | 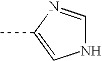 | 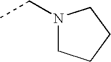 | H | 1.39 | 505.5 | 393 | A |
| 99 | 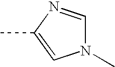 | 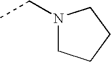 | H | 1.47 | 519.5 | 394 | A |
| 100 | 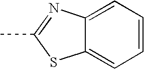 | 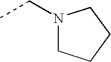 | H | 1.83 | 572.3 | 285 | A |
| 101 | 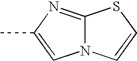 | 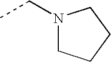 | H | 1.62 | 561.3 | 396 | A |
| 102 | 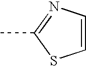 | 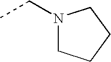 | H | 1.64 | 522.3 | 399 | A |
| 103 | 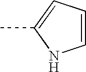 | 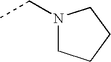 | H | 1.57 | 504.3 | 395 | A |
| 104 | 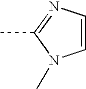 | 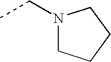 | H | 1.56 | 519.3 | 396 | A |
| 105 | 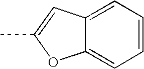 | 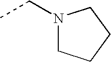 | H | 1.77 | 555.3 | 282 | A |
| 106 | 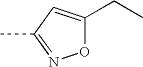 | 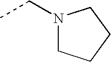 | H | 1.72 | 534.5 | 394 | A |
| 107 | 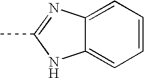 | 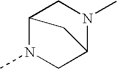 | H | 1.69 | 582.3 | 289 | A |
| 108 | 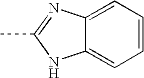 | 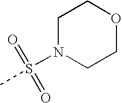 | H | 2.24 | 621.0 | 291 | A |
| 109 | 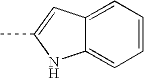 | 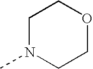 | H | 2.30 | 556.3 | 313 | A |
| 110 | 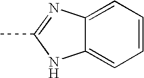 | 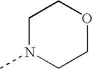 | H | 2.25 | 557.3 | 286 | A |
| 111 | 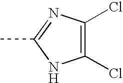 | 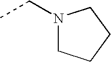 | H | 1.75 | 574.3 | 284 | A |
| 112 | 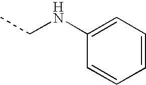 | 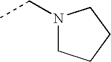 | H | 1.66 | 544.5 | 392 | A |
| 113 | 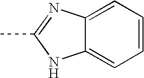 | 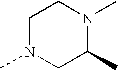 | H | 1.71 | 584.5 | 289 | A |
| 114 | 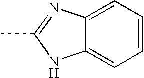 | 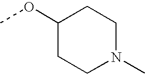 | H | 1.58 | 585.5 | 290 | A |
| 115 | 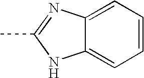 | 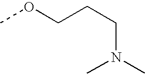 | H | 1.57 | 573.3 | 290 | A |
| 116 | 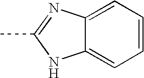 | 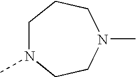 | H | 1.73 | 584.5 | 286 | A |
| 117 | 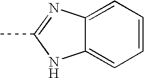 | 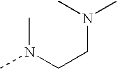 | H | 1.71 | 572.3 | 288 | A |
| 118 | 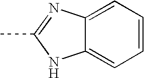 | 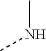 | H | 2.04 | 501.3 | 289 | A |
| 119 | 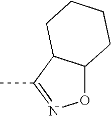 | 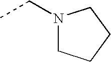 | H | 1.73 | 560.3 | 398 | A |
| 120 | 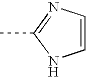 | 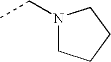 | H | 0.12 | 504.3 | 396 | A |
| 121 | 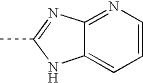 | 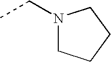 | H | 2.02 | 556.3 | 293 | B |
| 122 | 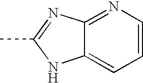 | 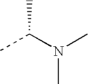 | H | 1.99 | 499.3 | 293 | B |
| 123 | 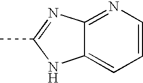 | 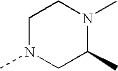 | H | 1.94 | 585.5 | 295 | B |
| 124 | 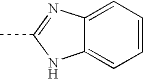 | 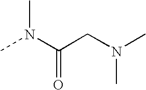 | H | 1.70 | 586.3 | 289 | A |
| 125 | 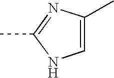 | 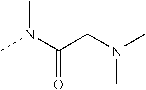 | H | 1.52 | 550.3 | 270 | A |
| 126 | 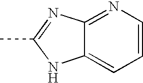 | 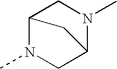 | H | 1.88 | 583.5 | 293 | B |

TABLE 2
Heteroarylmethylidene compounds

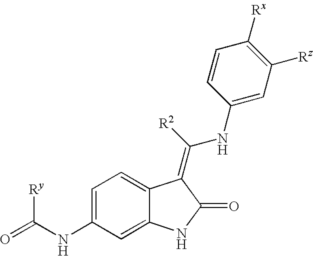

| | | | | | tᵣₑₜ | | UVₘₐₓ | HPLC |
|---|---|---|---|---|---|---|---|---|
| Ex. | R^{y} | R^{x} | R^{z} | R² | [min] | [M + H]⁺ | [nM] | Method |
| 127 | 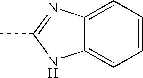 | 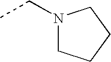 | H | 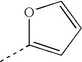 | 1.66 | 545.3 | 290 | A |
| 128 | 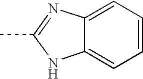 | 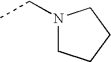 | H | 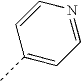 | 1.38 | 556.5 | 397 | A |
| 129 | 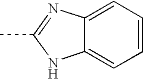 | 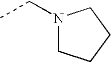 | H | 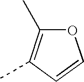 | 1.52 | 559.3 | 288 | A |
| 130 | 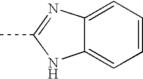 | 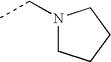 | H | 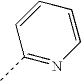 | 1.40 | 556.3 | 287 | A |
| 131 | 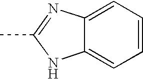 | 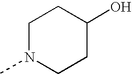 | H | 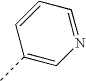 | 1.66 | 572.3 | 282 | A |
| 132 | 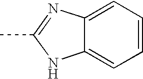 | 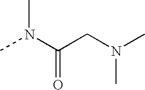 | H | 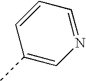 | 1.55 | 587.3 | 282 | A |
| 133 | 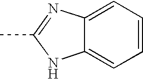 | 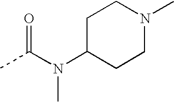 | H | 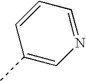 | 1.54 | 627.3 | 284 | A |
| 134 | 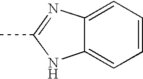 | 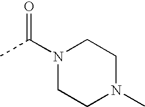 | H | 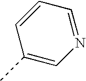 | 1.50 | 599.3 | 289 | A |
| 135 | 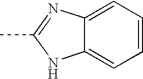 | 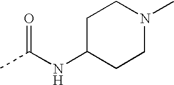 | H | 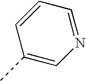 | 1.54 | 613.3 | 289 | A |
| 136 | 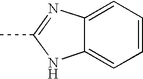 | 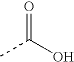 | H | 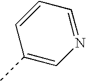 | 1.90 | 517.3 | 290 | A |
| 137 | 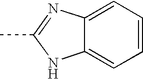 | 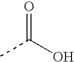 | Cl | 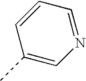 | 1.96 | 551.2 | 291 | A |
| 138 | 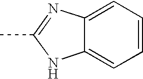 | 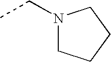 | H | 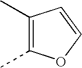 | 1.68 | 559.3 | 289 | A |
| 139 | 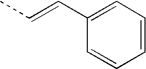 | 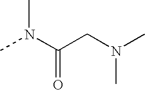 | H | 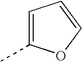 | 1.71 | 562.3 | 286 | A |
| 140 | 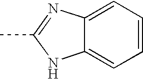 | 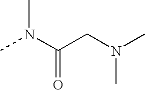 | H | 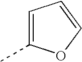 | 1.61 | 576.2 | 290 | A |
| 141 | 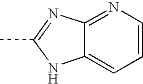 | 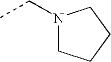 | H | 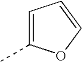 | 1.96 | 546.3 | 295 | B |
| 142 | 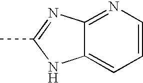 | 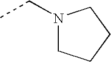 | H | 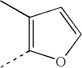 | 1.98 | 560.3 | 294 | B |
| 143 | 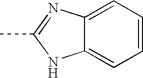 | 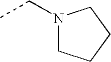 | H | 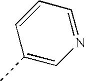 | 1.39 | 556.3 | 284 | A |

TABLE 3
Variation at the aniline

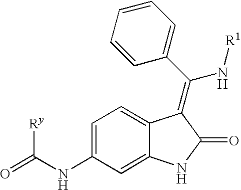

| | | | tᵣₑₜ | | UVₘₐₓ | HPLC |
|---|---|---|---|---|---|---|
| Ex. | R^{y} | R¹ | [min] | [M + H]⁺ | [nM] | Method |
| 144 | 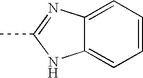 | - - - -H | 1.971 | 396.3 | 371 | A |
| 145 | 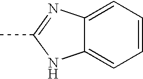 | 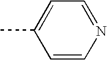 | 1.601 | 473.3 | 298 | A |
| 146 | 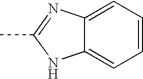 | 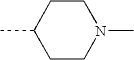 | 1.57 | 493.3 | 375 | A |
| 147 | 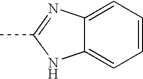 | 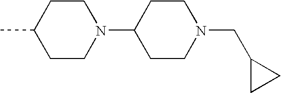 | 1.432 | 616.5 | 374 | A |
| 148 | 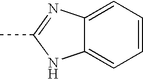 | 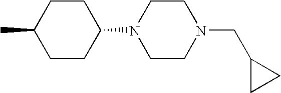 | 1.574 | 616.3 | 375 | A |
| 149 | 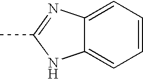 | - - - - | 2.12 | 409.3 | 371 | A |
| 150 | 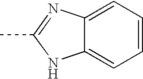 | - - - - | 2.059 | 410.3 | 376 | A |
| 151 | 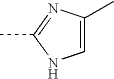 | - - - - | 1.74 | 374.3 | 372 | A |
| 152 | 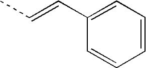 | 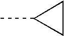 | 2.25 | 422.2 | 282 | A |
| 153 | 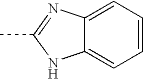 | 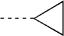 | 2.194 | 436.3 | 380 | A |
| 154 | 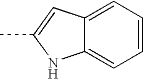 | 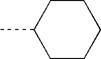 | 2.46 | 477.2 | 373 | A |
| 155 | 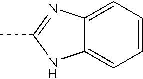 | 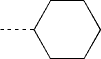 | 2.426 | 478.3 | 377 | A |
| 156 | 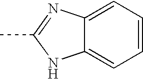 | 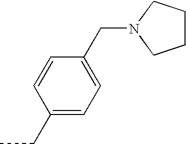 | 1.72 | 569.3 | 283 | A |
| 157 | 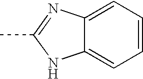 | 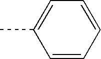 | 2.29 | 472.3 | 289 | B |
| 158 | 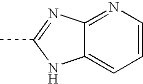 | 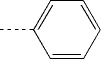 | 2.01 | 473.3 | 292 | B |
| 159 | 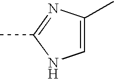 | 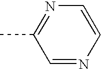 | 1.87 | 438.3 | 298 | B |
| 160 | 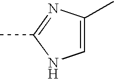 | 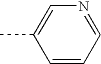 | 1.88 | 451.2 | 296 | B |
| 161 | 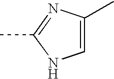 | 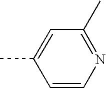 | 1.87 | 451.2 | 296 | B |
| 162 | 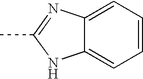 | 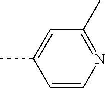 | 2.06 | 487.3 | 289 | B |
| 163 | 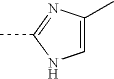 | 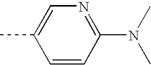 | 1.97 | 480.3 | 390 | B |
| 164 | 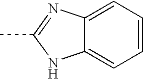 | 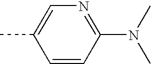 | 2.16 | 516.3 | 395 | B |
| 165 | 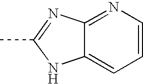 | 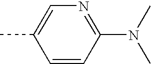 | 1.87 | 517.3 | 395 | B |
| 166 | 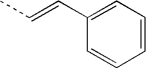 | 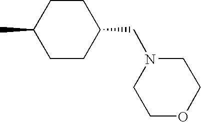 | 1.71 | 563.3 | 378 | A |
| 167 | 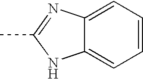 | 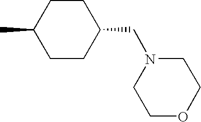 | 1.671 | 577.3 | 376 | A |
| 168 | 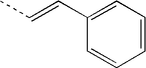 | 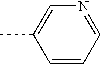 | 2.08 | 459.2 | 284 | B |
| 169 | 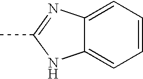 | 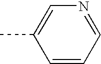 | 2.02 | 473.3 | 289 | B |
| 170 | 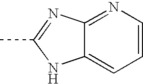 | 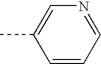 | 1.71 | 474.3 | 292 | B |
| 171 | 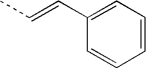 | 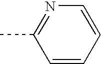 | 2.22 | 459.2 | 284 | B |
| 172 | 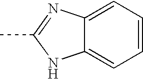 | 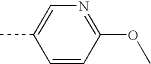 | 2.16 | 503.3 | 287 | B |
| 173 | 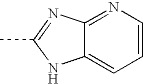 | 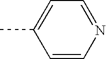 | 1.70 | 474.3 | 292 | B |

TABLE 4
Bisheteroarylindolinones

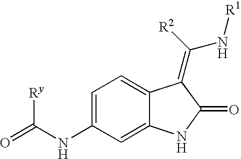

| | | | | tᵣₑₜ | | UVₘₐₓ | HPLC |
|---|---|---|---|---|---|---|---|
| Ex. | R^{y} | R² | R¹ | [min] | [M + H]⁺ | [nM] | Method |
| 174 | 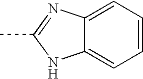 | 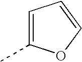 | 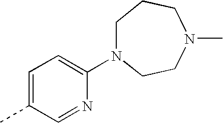 | 1.61 | 575.3 | 286 | A |

TABLE 5
Pyridylamines

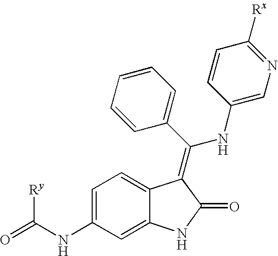

| | | | tᵣₑₜ | | UVₘₐₓ | HPLC |
|---|---|---|---|---|---|---|
| Ex. | R^{y} | R^{x} | [min] | [M + H]⁺ | [nM] | Method |
| 175 | 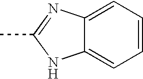 | 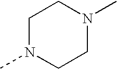 | 1.66 | 571.3 | 287 | A |
| 176 | 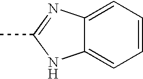 | 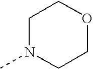 | 2.06 | 558.3 | 285 | A |
| 177 | 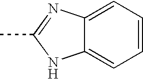 | 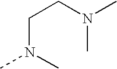 | 1.69 | 573.3 | 287 | A |
| 178 | 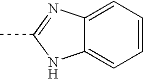 | 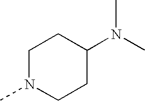 | 1.64 | 599.3 | 287 | A |
| 179 | 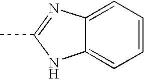 | 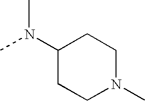 | 1.59 | 599.3 | 288 | A |
| 180 | 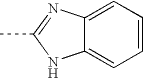 | 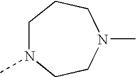 | 1.65 | 585.3 | 286 | A |
| 181 | 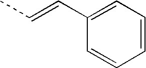 | 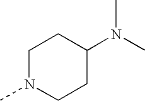 | 1.69 | 585.3 | 284 | A |
| 182 | 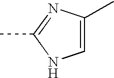 | 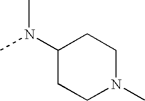 | 0.12 | 563.3 | 388 | A |
| 183 | 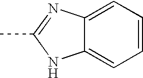 | 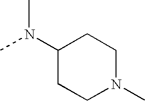 | 1.59 | 599.3 | 396 | A |
| 184 | 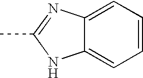 | 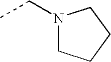 | 2.03 | 556.3 | 289 | B |
| 185 | 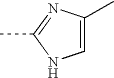 | 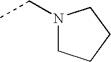 | 1.90 | 520.3 | 298 | B |

### Preparation of Substituted Acetamide Derivatives

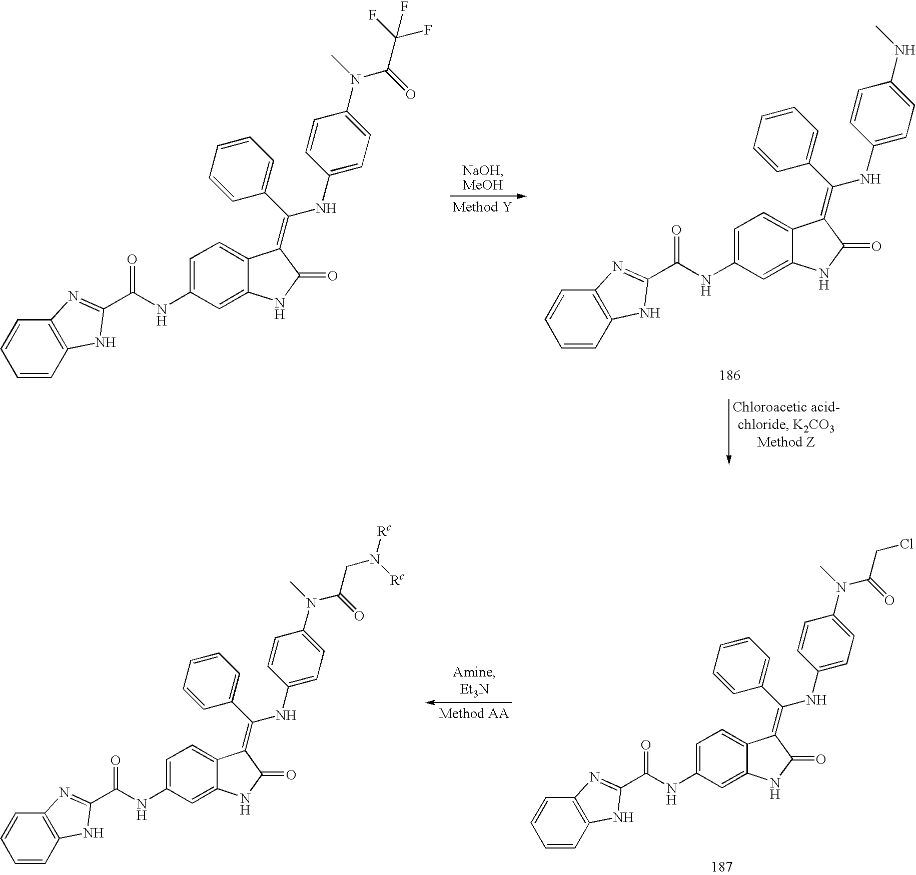

### Method Y—Cleaving the Trifluoracetyl Protective Group

The trifluoracetamide (4.39 g, 6.87 mmol) is suspended in MeOH (30 mL)/2 N NaOH (18 mL) and stirred for 2 h. The mixture is diluted with 25% EtOH the precipitate is isolated by filtration, washed with water and the solid is dried in vacuo. Yield: 2.80 g (81%) (186).

Method Z—Reaction with Chloroacetic Acid Chloride

Chloroacetic acid chloride (300 μL) is added to 186 (800 mg, 1.60 mmol) and K₂CO₃ (450 mg, 3.22 mmol) in anhydrous CH₂Cl₂ (10 mL) and stirred for 16 h at RT. The reaction mixture is washed with saturated NaHCO₃ solution and saturated NaCl solution, dried, filtered and evaporated down. Yield: 900 mg (98%) (187).

Method AA—Reaction with Primary and Secondary Amines

187 (50 mg, 87 μmmol), pyrrolidine (10.8 μt, 130 μmol) and Et₃N (60 μL) are stirred in anhydrous NMP (0.5 mL) in the microwave for 6 min at 150° C. The reaction mixture is filtered and purified by preparative HPLC.

| | | | | Yield |
|---|---|---|---|---|
| Ex. | Structure | Educt | Method | [%] |
| 188 | 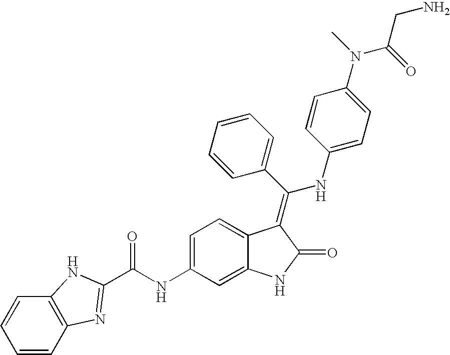 | 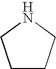 | AA | 62 |
| 189 | 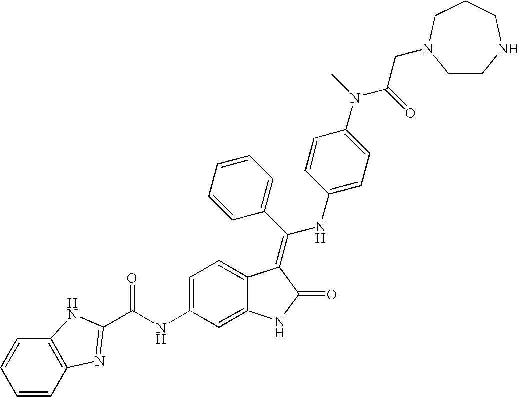 | 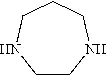 | AA | 39 |
| 190 | 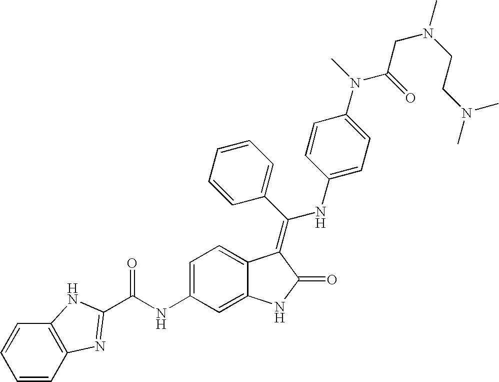 | 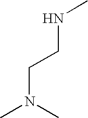 | AA | 36 |
| 191 | 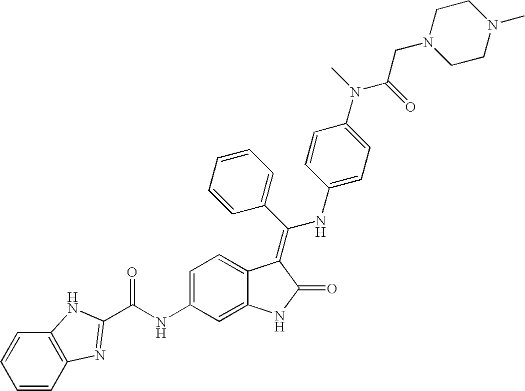 | 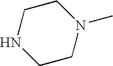 | AA | 59 |
| 192 | 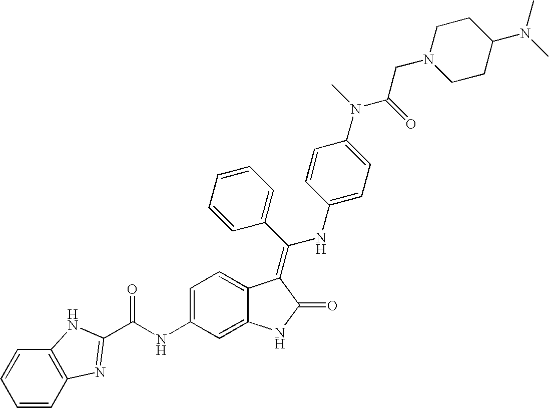 | 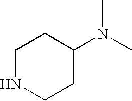 | AA | 65 |
| 193 | 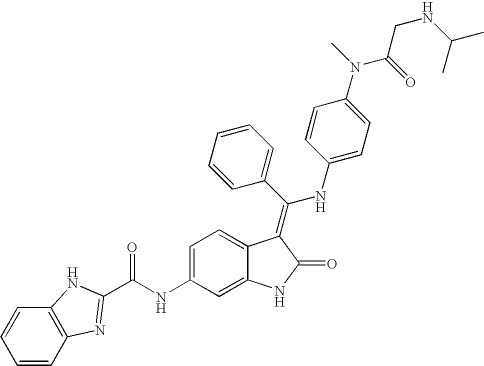 | 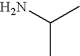 | AA | 38 |

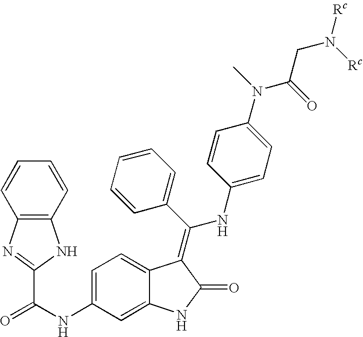

| | | tᵣₑₜ | | UVₘₐₓ | HPLC- |
|---|---|---|---|---|---|
| Ex. | NR^{c}R^{c} | [min] | [M + H]⁺ | [nM] | Method |
| 194 | 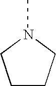 | 1.72 | 612.3 | 289 | A |
| 195 | 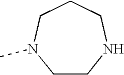 | 1.57 | 641.3 | 289 | A |
| 196 | 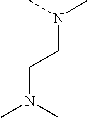 | 1.68 | 643.3 | 289 | A |
| 197 | 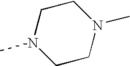 | 1.68 | 641.3 | 289 | A |
| 198 | 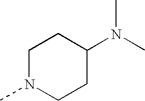 | 1.54 | 669.2 | 289 | A |
| 199 | 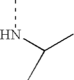 | 1.67 | 627.3 | 289 | A |

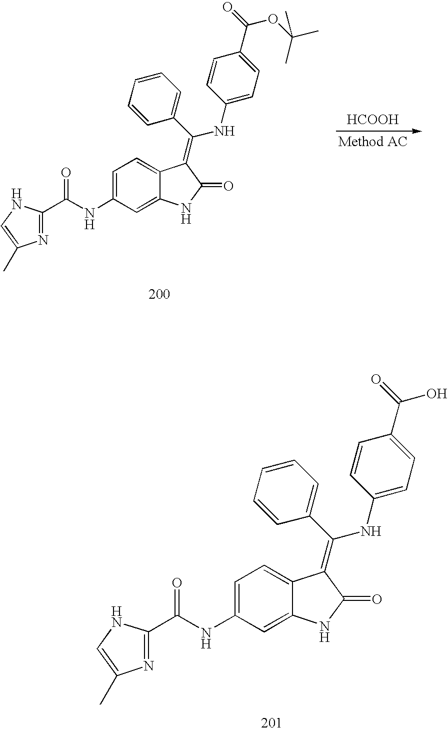

### Method AC—Ester Cleaving

The ester (200) (203 mg, 0.38 mmol) is stirred in formic acid (4 mL) for 2 h at 50° C. The mixture is evaporated down and the residue is recrystallised from MeOH. Yield: 111 mg (61%). If no crystalline product is obtained, the crude mixture is purified by preparative HPLC.

| | | | | Yield |
|---|---|---|---|---|
| Ex. | Structure | Educt | Method | [%] |
| 201 | 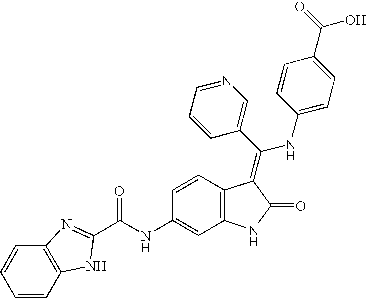 | 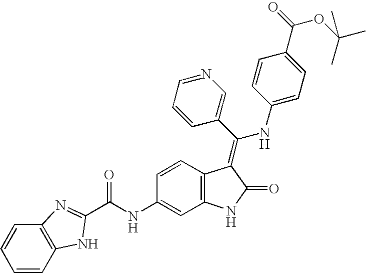 | AC | quant. |
| 202 | 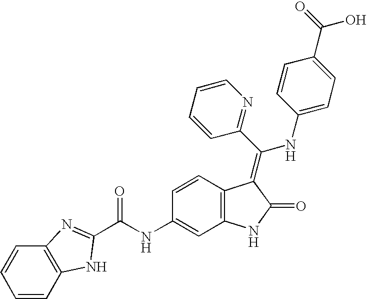 | 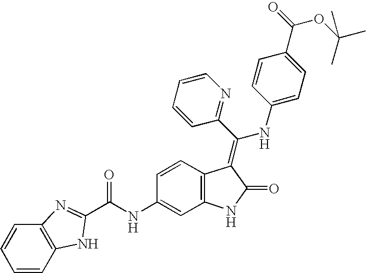 | AC | quant. |
| 203 | 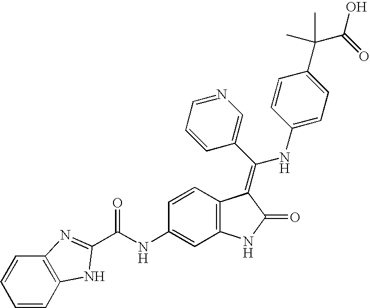 | 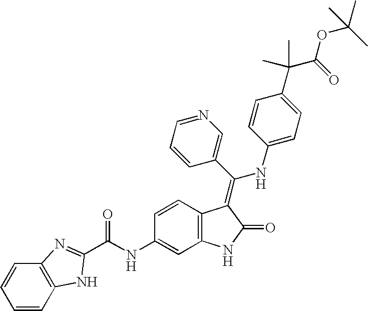 | AC | 97 |
| 204 | 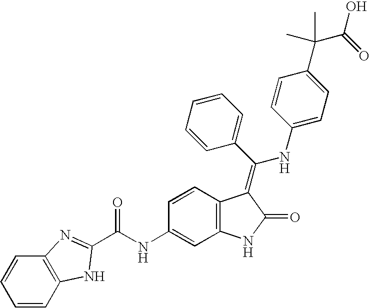 | 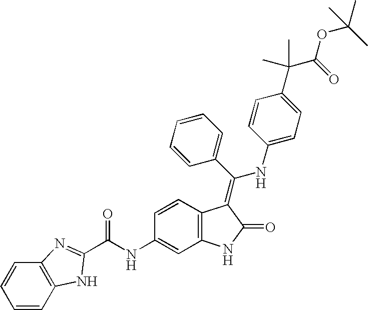 | AC | 94 |
| 205 | 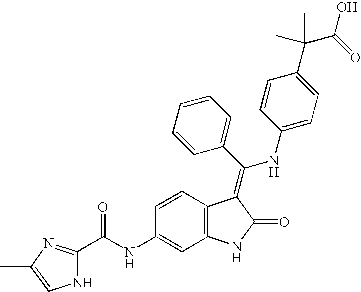 | 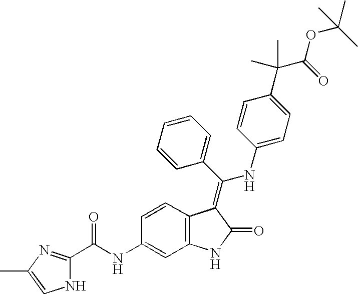 | AC | 79 |

### Abbreviations Used

Ac acetyl
Bu butyl
DCM dichloromethane

### DMF N,N-dimethylformamide

DMSO dimethylsulphoxide
DTT dithiothreitol
EDTA ethylene diamine tetraacetic acid
equiv equivalent
Et ethyl
EtOAc ethyl acetate
h hour
HPLC high performance liquid chromatography
conc. concentrated
HMDS hexamethyldisilazane
iPrOH isopropanol
Me methyl
MeOH methanol
min minute
mL millilitre
MS mass spectrometry
N normal

### NMP N-methylpyrrolidinone

NMR nuclear magnetic resonance spectroscopy
PBS phosphate buffered saline
ppm part per million
RP reversed phase
RT ambient temperature
TFA trifluoro acetic acid
TBTU O-benzotriazol-1-yl-N,N,N′,N′-tetramethyluronium tetrafluoroborate
tent tertiary
THF tetrahydrofuran
TMSCl chlorotrimethylsilane

### HPLC Methods

### HPLC: Agilent 1100 Series

MS: Agilent LC/MSD SL (LCMS1: 1100 series LC/MSD)
Column: Waters, Xterra MS C18, 2.5 μm, 2.1×30 mm, Part. No. 186000592
Solvent: A: H₂O (Millipore purified purest water) with 0.1% HCOOH
B: acetonitrile (HPLC grade)
   Detection: MS: Positive and negative
Mass range: 120-900 m/z
Fragmentor: 120
Gain EMV: 1
Threshold: 150
Stepsize: 0.25
UV: 254 nm
Bandwide: 1 (LCMS1: 2)
Reference: off

### Spectrum: Range: 250-400 nm

Range step: 1.00 nm
Threshold: 4.00 mAU
Peakwidth: <0.01 min (LCMS1: >0.05 min)
Slit: 1 nm (LCMS1: 2 nm)
   Injection: Inj. Vol.: 5 μL
   Inj. mode: Needle wash
   Separation: Flow: 1.10 mL/min
Column temp.: 40° C.
Gradient: 0 min 5% solvent B
   0-2.5 min 5% ->95% solvent B
   2.50-2.80 min 95% solvent B
   2.81-3.10 min 95% ->5% solvent B

### Method B

HPLC: Agilent 1100 Series
MS: 1100 Series LC/MSD (API-ES +/− 3000 V, Quadrupol, G1946D)
MSD Signal Settings: Scan pos 120-900, Scan neg 120-900
Column: Phenomenex; Part No. 00M-4439-BO-CE; Gemini 3μ
C18 110 Å; 20 × 2.0 mm column
Eluant:
A: 5 mM NH₄HCO₃/20 mM NH₃ (pH = 9.5)
B: acetonitrile HPLC grade
Detection:
SignaL: UV 254 nm (bandwide 1, reference off)
Spectrum: range: 250-400 nm; step: 1 nm
Peak width <0.01 min (0.1 s)
Injection: 10 μl standard injection
Method: LCMSBAS1
flow: 1.0 ml/min
column temp.: 40° C.

| | | |
|---|---|---|
| pump gradient: | 0.0-2.5 min | 5% −> 95% solvent B |
| | 2.5-2.8 min | 95% solvent B |
| | 2.8-3.1 min | 95% −> 5% solvent B |

The Examples describe the biological activity of the compounds according to the invention without restricting the invention to these Examples.

As demonstrated by DNA staining followed by FACS or Cellomics Array Scan analysis, the inhibition of proliferation brought about by the compounds according to the invention is mediated above all by errors in chromosome segregation. Because of the accumulation of faulty segregations, massive polyploidia occurs which may finally lead to inhibition of proliferation or even apoptosis. On the basis of their biological properties the compounds of general formula (I) according to the invention, their isomers and the physiologically acceptable salts thereof are suitable for treating diseases characterised by excessive or abnormal cell proliferation.

### Example Aurora-B Kinase Assay

A radioactive enzyme inhibition assay was developed using E. coli-expressed recombinant Xenopus laevis Aurora B wild-type protein equipped at the N-terminal position with a GST tag (amino acids 60-361) in a complex with Xenopus laevis INCENP (amino acids 790-847), which is obtained from bacteria and purified. In equivalent manner a Xenopus laevis Aurora B mutant (G96V) in a complex with Xenopus laevis INCENP⁷⁹⁰⁻⁸⁴⁷ may also be used.

### Expression and Purification

The coding sequence for Aurora-B⁶⁰⁻³⁶¹ from Xenopus laevis is cloned into a modified version of pGEX-6T (Amersham Biotech) via BamHI and SalI cutting sites. The vector contains two cloning cassettes which are separated by a ribosomal binding site, allowing bi-cistronic expression. In this configuration Xenopus laevis Aurora B is expressed by the first cassette, and the Xenopus laevis INCENP⁷⁹⁰⁻⁸⁴⁷ is expressed by the second cassette. The resulting vector is pAUB-IN⁸⁴⁷.

First of all the E. coli strain BL21 (DE3) is co-transformed with pUBS520 helper plasmid and pAUB-1N⁸⁴⁷, after which protein expression is induced using 0.3 mM IPTG at an OD₆₀₀ of 0.45-0.7. The expression is then continued for approx. 12-16 h at 23-25° C. with agitation.

The bacteria are then removed by centrifuging and the pellet is lysed in lysis buffer (50 mM Tris/C1 pH 7.6, 300 mM NaCl, 1 mM DTT, 1 mM EDTA, 5% glycerol, Roche Complete Protease Inhibitor tablets) using ultrasound, using 20-30 mL lysis buffer per litre of E. coli culture. The lysed material is freed from debris by centrifugation (12000 rpm, 45-60 min, JA20 rotor). The supernatant is incubated with 300 μL of equilibrated GST Sepharose Fast Flow (Amersham Biosciences) per litre of E. coli culture for 4-5 h at 4° C. Then the column material is washed with 30 volumes of lysis buffer and then equilibrated with 30 volumes of cleavage buffer (50 mM Tris/C1 pH 7.6, 150 mM NaCl, 1 mM DTT, 1 mM EDTA). To cleave the GST tag from Aurora B, 10 units of Prescission Protease (Amersham Biosciences) are used per milligram of substrate and the mixture is incubated for 16 h at 4° C. The supernatant which contains the cleavage product is loaded onto a 6 mL Resource Q column (Amersham Biosciences) equilibrated with ion exchange buffer (50 mM Tris/C1 pH 7.6, 150 mM NaCl, 1 mM DTT, 1 mM EDTA). The Aurora B/INCENP complex is caught as it flows through, then concentrated and loaded onto a Superdex 200 size exclusion chromatography (SEC) column equilibrated with SEC buffer (10 mM Tris/C1 pH 7.6, 150 mM NaCl, 1 mM DTT, 1 mM EDTA). Fractions which contain the AuroraB/INCENP complex are collected and concentrated using Vivaspin concentrators (molecular weight exclusion 3000-5000 Da) to a final concentration of 12 mg/mL. Aliquots (e.g. 240 ng/μL) for kinase assays are transferred from this stock solution into freezing buffer (50 mM Tris/Cl pH 8.0, 150 mM NaCl, 0.1 mM EDTA, 0.03% Brij-35, 10% glycerol, 1 mM DTT) and stored at −80° C.

### Kinase Assay

Test substances are placed in a polypropylene dish (96 wells, Greiner #655 201), in order to cover a concentration frame of 10 μM-0.0001 μM. The final concentration of DMSO in the assay is 5%. 30 μL of protein mix (50 mM tris/C1 pH 7.5, 25 mM MgCl₂, 25 mM NaCl, 167 μM ATP, 10 ng Xenopus laevis Aurora B/INCENP complex in freezing buffer) are pipetted into the 10 μl of test substance provided in 25% DMSO and this is incubated for 15 min at RT. Then 10 μL of peptide mix (100 mM tris/C1 pH 7.5, 50 mM MgCl₂, 50 mM NaCl, 5 μM NaF, 5 μM DTT, 1 μCi gamma-P33-ATP [Amersham], 50 μM substrate peptide [biotin-EPLERRLSLVPDS or multimers thereof, or biotin-EPLERRLSLVPKM or multimers thereof, or biotin-LRRWSLGLRRWSLGLRRWSLGL RRWSLG]) are added. The reaction is incubated for 75 min (ambient temperature) and stopped by the addition of 180 μL of 6.4% trichloroacetic acid and incubated for 20 min on ice. A multiscreen filtration plate (Millipore, MAIP NOB10) is equilibrated first of all with 100 μL 70% ethanol and then with 180 μL trichloroacetic acid and the liquids are eliminated using a suitable suction apparatus. Then the stopped kinase reaction is applied. After 5 washing steps with 180 μL 1% trichloroacetic acid in each case the lower half of the dish is dried (10-20 min at 55° C.) and 25 μL scintillation cocktail (Microscint, Packard # 6013611) is added. Incorporated gamma-phosphate is quantified using a Wallac 1450 Microbeta Liquid Scintillation Counter. Samples without test substance or without substrate peptide are used as controls. IC₅₀ values are obtained using Graph Pad Prism software.

The anti-proliferative activity of the compounds according to the invention is determined in the proliferation test on cultivated human tumour cells and/or in a cell cycle analysis, for example on NCI-H460 tumour cells. In both test methods compounds 1-205 exhibit good to very good activity, i.e. for example an EC50 value in the NCI-H460 proliferation test of less than 5 μmol/L, generally less than 1 μmol/L.

### Measurement of the Inhibition of Proliferation on Cultivated Human Tumour Cells

To measure proliferation on cultivated human tumour cells, cells of lung tumour cell line NCI-H460 (obtained from American Type Culture Collection (ATCC)) are cultivated in RPMI 1640 medium (Gibco) and 10% foetal calf serum (Gibco) and harvested in the log growth phase. Then the NCI-H460 cells are placed in 96-well flat-bottomed plates (Falcon) at a density of 1000 cells per well in RPMI 1640 medium and incubated overnight in an incubator (at 37° C. and 5% CO₂). The active substances are added to the cells in various concentrations (dissolved in DMSO; DMSO final concentration: 0.1%). After 72 hours incubation 20 μl AlamarBlue reagent (AccuMed International) is added to each well, and the cells are incubated for a further 5-7 h. After incubation the colour change of the AlamarBlue reagent is determined in a Wallac Microbeta fluorescence spectrophotometer.

EC₅₀ values are calculated using Standard Levenburg Marquard algorithms (GraphPadPrizm).

Cell cycle analyses are carried out for example using FACS analyses (Fluorescence Activated Cell Sorter) or by Cellomics Array Scan (CellCycle Analysis).

### FACS Analysis

Propidium iodide (PI) binds stoichiometrically to double-stranded DNA, and is thus suitable for determining the proportion of cells in the G1, S, and G2/M phase of the cell cycle on the basis of the cellular DNA content. Cells in the G0 and G1 phase have a diploid DNA content (2N), whereas cells in the G2 or mitosis phase have a 4N DNA content.

For PI staining, for example, 1.75×10⁶ NCI-H460 cells are seeded onto a 75 cm² cell culture flask, and after 24 h either 0.1% DMSO is added as control or the substance is added in various concentrations (in 0.1% DMSO). The cells are incubated for 42 h with the substance or with DMSO. Then the cells are detached with trypsin and centrifuged. The cell pellet is washed with buffered saline solution (PBS) and the cells are then fixed with 80% ethanol at −20° C. for at least 2 h. After another washing step with PBS the cells are permeabilised with Triton X-100 (Sigma; 0.25% in PBS) on ice for 5 min, and then incubated with a solution of PI (Sigma; 10 μg/ml)and RNAse (Serva; 1 mg/mL1) in the ratio 9:1 for at least 20 min in the dark.

The DNA measurement is carried out in a Becton Dickinson FACS Analyzer, with an argon laser (500 mW, emission 488 nm); data are obtained and evaluated using the DNA Cell Quest Programme (BD).

### Cellomics Array Scan

NCI-H460 cells are seeded into 96-well flat-bottomed dishes (Falcon) in RPMI 1640 medium (Gibco) with 10% foetal calf serum (Gibco) in a density of 2000 cells per well and incubated overnight in an incubator (at 37° C. and 5% CO₂). The active substances are added to the cells in various concentrations (dissolved in DMSO; DMSO final concentration: 0.1%). After 42 h incubation the medium is suction filtered, the cells are fixed for 10 min with 4% formaldehyde solution and Triton X-100 (1:200 in PBS) at ambient temperature and simultaneously permeabilised, and then washed twice with a 0.3% BSA solution (Calbiochem). Then the DNA is stained by the addition of 50 μl/well of 4′,6-diamidino-2-phenylindole (DAPI; Molecular Probes) in a final concentration of 300 nM for 1 h at ambient temperature, in the dark. The preparations are then carefully washed twice with PBS, the plates are stuck down with black adhesive film and analysed in the Cellomics ArrayScan using the CellCycle BioApplication programme and visualised and evaluated using Spotfire.

The substances of the present invention are Aurora kinase inhibitors. On the basis of their biological properties the compounds of general formula (I) according to the invention, their isomers and the physiologically acceptable salts thereof are suitable for treating diseases characterised by excessive or abnormal cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours (e.g. carcinomas and sarcomas), skin diseases (e.g. psoriasis); diseases based on hyperplasia which are characterised by an increase in the number of cells (e.g. fibroblasts, hepatocytes, bones and bone marrow cells, cartilage or smooth muscle cells or epithelial cells (e.g. endometrial hyperplasia)); bone diseases and cardiovascular diseases (e.g. restenosis and hypertrophy).

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supraglottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, giant cell tumour, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the oesophagus; penile cancer; prostate cancer; throat cancer or carcinomas of the pharynx such as for example nasopharynx carcinomas, oropharynx carcinomas and hypopharynx carcinomas; retinoblastoma, vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva.

The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (1) may be used on their own or in combination with other active substances according to the invention, optionally also in combination with other pharmacologically active substances.

Chemotherapeutic agents which may be administered in combination with the compounds according to the invention, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortinsone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor” and “hepatocyte growth factor”, inhibitors are for example “growth factor” antibodies, “growth factor receptor” antibodies and tyrosinekinase inhibitors, such as for example gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions,—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered.

Thus, in some cases it may be sufficient to use less than the minimum dose given above, whereas in other cases the upper limit may have to be exceeded. When administering large amounts it may be advisable to divide them up into a number of smaller doses spread over the day.

The formulation examples which follow illustrate the present invention without restricting its scope:

### Examples of Pharmaceutical Formulations

| | | |
|---|---|---|
| A) | Tablets | per tablet |

| | |
|---|---|
| active substance according to formula (1) | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| | | |
|---|---|---|
| B) | Tablets | per tablet |

| | |
|---|---|
| active substance according to formula (1) | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

| | |
|---|---|
| C) | Ampoule solution |

| | |
|---|---|
| active substance according to formula (1) | 50 mg |
| sodium chloride | 50 mg |
| water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of formula (1), 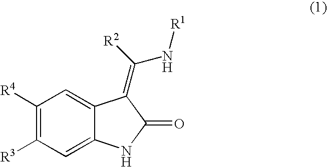
wherein
R¹ denotes hydrogen or a group, optionally substituted by one or more R⁵, selected from among C₃₋₁₀cycloalkyl, 3-8 membered heterocycloalkyl, C₆₋₁₅aryl and 5-15 membered heteroaryl; and
R² denotes a group, optionally substituted by one or more R⁵, selected from among C₆₋₁₅aryl and 5-15 membered heteroaryl; and
R³ denotes a group, optionally substituted by one or more R⁵, selected from among 3-8 membered heterocycloalkyl and 5-12 membered heteroaryl, or —N(R^{g})C(O)R^{c}, —N(R^{g})S(O)₂R^{c}, —N(R^{g})S(O)₂NR^{c}R^{c}, N(R^{g})[C(O)]₂NR^{c}R^{c}, —N(R^{g})C(O)OR^{c}, and
R⁴ denotes hydrogen or a group selected from among halogen, —CN, —OR^{e}, —NR^{e}R^{e} and C₁₋₆alkyl, and
R⁵ in each case independently of one another denote a group selected from among R^{a}, R^{b} and R^{a} substituted by one or more identical or different R^{b} and/or R^{c}; and
each R^{a} independently of one another is selected from among C₁₋₆alkyl, C₃₋₁₀cycloalkyl, C₄₋₁₆cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl;
each R^{b} is a suitable group and each independently selected from among ═O, —OR^{c}, C₁₋₃haloalkyloxy, —OCF₃, ═S, —SR^{C}, ═NR^{C}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{C}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N{[C(O)]₂R^{c}}₂, —N(R^{g})[C(O)]₂OR^{c}, —N(R^{g})[C(O)]₂NR^{c}R^{c}, —N{[C(O)]₂OR^{c}}₂, —N{[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c},
each R^{c} independently of one another denotes hydrogen or a group optionally substituted by one or more identical or different R^{d} and/or R^{e} selected from among C₁₋₆alkyl, C₃₋₁₀cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl;
each R^{d} is a suitable group and each independently selected from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}—N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e}}₂, —N(R^{g})[C(O)]₂OR^{e}, —N(R^{g})[C(O)]₂NR^{e}R^{e}, —N{[C(O)]₂OR^{e}}₂, —N{[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e} and —N(R^{g})C(NR^{g})NR^{e}R^{e},
each R^{e} independently of one another denotes hydrogen or a group optionally substituted by one or more identical or different R^{f} and/or R^{g} selected from among C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkylalkyl, 5-12 membered heteroaryl and 6-18 membered heteroarylalkyl;
each R^{f} is a suitable group and each independently selected from among halogen and —CF₃; and
each R^{g} independently of one another denotes hydrogen, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-8 membered heterocycloalkyl, 4-14 membered heterocycloalkyl, 5-12 membered heteroaryl or 6-18 membered heteroarylalkyl,
a tautomer thereof, a racemate thereof, an enantiomer thereof, or a diastereomer thereof, mixtures of any of the foregoing, or a pharmacologically acceptable acid addition salt thereof, with the proviso that 6-benzoylamino-3-(Z)-{1-[4-(piperidin-1yl-methyl)-anilino]-1-phenyl-methylidene}-2-indolinone, 3-(Z)-{1-[4-(piperidin-1-yl-methyl)-anilino]-1-phenyl-methylidene}-6-(pyrrol-1-yl)-2-indolinone and 3-(Z)-{1-[4-(piperidin-1-yl-methyl)-anilino]-1-phenyl-methylidene}-6-(pyrrolidin-1-yl)-2-indolinone are not included.

2. The compound according to claim 1, wherein R⁴ is hydrogen.

3. The compound according to claim 1, wherein R¹ denotes phenyl.

4. The compound according to claim 1, wherein R² denotes phenyl.

5. The compound according to claim 4, wherein R² denotes unsubstituted phenyl.

6. The compound according to claim 1, wherein R³ denotes —N(R^{g})C(O)R^{c}.

7-8. (canceled)

9. A pharmaceutical preparation, comprising as active substance one or more compounds of formula (1) according to claim 1 in combination with one or more conventional excipients and/or carriers.

10. (canceled)

11. A pharmaceutical preparation comprising a compound of formula (1) according to claim 1 and at least one further cytostatic or cytotoxic active substance, different from formula (1).

12. A method for the treatment or prevention of cancer, infections, inflammations or autoimmune disease which comprises administering a therapeutically effective amount of one or more compounds according to claim 1.
